(19) United States
(12) Patent Application Publication
(10) Pub. No.: US 2010/0076269 A1
(43) Pub. Date: Mar. 25, 2010

(54) Systems and Methods for Treating Sinuses

(75) Inventors: Joshua Makower, Los Altos, CA (US); John Y. Chang, Mountain View, CA (US); William M. Facteau, Mountain View, CA (US); Eric Goldfarb, Belmont, CA (US); Ketan P. Muni, San Jose, CA (US); Earl A. Bright, II, Los Altos, CA (US); Greg Garfield, Los Gatos, CA (US); Greg Liu, Sunnyvale, CA (US); Thomas Jenkins, Oakland, CA (US); John H. Morriss, San Francisco, CA (US); Julia D. Vrany, Sunnyvale, CA (US); Hung V. Ha, San Jose, CA (US); Ronda M. Heiser, San Jose, CA (US); Scott M. Smith, Menlo Park, CA (US)

Correspondence Address: STEPTOE & JOHNSON - ACCLARENT, INC., 2121 AVENUE OF THE STARS, SUITE 2800, LOS ANGELES, CA, 90067, US

(73) Assignee: ACCLARENT, INC., Menlo Park, CA (US)

(21) Appl. No.: 12/562,828

(22) Filed: Sep. 18, 2009

Related U.S. Application Data
(60) Provisional application No. 61/098,080, filed on Sep. 18, 2008.

(51) Int. Cl. A61M 29/02; A61B 6/12; A61M 25/10; A61B 17/24; A61B 1/06; A61B 17/32

(52) U.S. Cl. 600/178; 606/199; 600/424; 604/509; 606/110; 606/170

(57) ABSTRACT

Systems and methods for treating paranasal sinuses in a head of a patient include, for example, forming an opening through a canine fossa into a maxillary paranasal sinus and performing a procedure such as a balloon catheter dilation of the maxillary sinus ostium. In some embodiments, one or more procedures other than a balloon dilation procedure may be performed. In some embodiments, a combination of balloon dilation and one or more other procedures may be performed. Various approaches involve employing medical devices to accomplish alternative treatment modalities as well as taking alternative routes to the interventional site.

### CROSS-REFERENCES TO RELATED APPLICATIONS

This application claims the benefit of Provisional Application Ser. No. 61/098,080, filed Sep. 18, 2008, the contents of which are incorporated by reference.

### BACKGROUND

The present invention relates generally to medical devices and methods. More specifically, the present invention relates to medical devices, systems and methods for treating paranasal sinuses, such as in patients suffering from chronic or acute recurring sinusitis.

Sinusitis is a condition affecting over 35 million Americans and similarly large populations in the rest of the developed world. Sinusitis occurs when one or more of the four pairs of paranasal sinus cavities (i.e., maxillary, ethmoid, frontal, sphenoid) becomes obstructed. The paranasal sinuses are part of the skull and are composed of bone covered with mucous membrane (or “mucosa”). They are located in the skull behind the face—maxillary sinuses behind the cheeks, frontal sinuses in the forehead, ethmoid sinuses between/behind the eyes and sphenoid sinuses farther back in the skull. (The sinuses are depicted in FIGS. 1 and 2, which are described in the Detailed Description below.) Normally, the sinuses produce mucus, which is then moved by beating cilia from the sinus cavities through openings (or “ostia,” singular “ostium”) into the nasal cavity and down the throat. The combined sinuses produce approximately one litre of mucus daily, so the effective transport of this mucus is important to sinus health. When the mucosa of one or more ostia or regions near the ostia become inflamed, the egress of mucus is interrupted, setting the stage for an infection of the sinus cavity, i.e., sinusitis.

Though acute sinusitis may be treatable with antibiotics and/or anti-inflammatory nasal sprays such as steroids, in some cases sinusitis persists for months, a condition called chronic sinusitis. Some patients are also prone to multiple episodes of sinusitis in a given period of time, a condition called acute recurrent sinusitis.

Currently, chronic sinusitis patients may elect to have a surgical procedure called functional endoscopic sinus surgery (FESS). In this procedure, typically performed in an operating room with the patient under general anesthesia, an ear, nose and throat (ENT) surgeon uses surgical cutting instruments, guided with endoscopic visualization, to remove bone and mucosal from the nasal cavity and widen the paranasal sinus ostia. In many cases, the ethmoid sinuses are simply removed with the cutting tools. Inflamed mucosa and underlying bony tissue are cut away in an effort to widen the outlet of the sinuses of interest. Once opened, the infected sinuses are able to drain and return to a relatively normal state.

While FESS is often effective, it is a relatively invasive procedure, typically accompanied by significant post-operative pain and bleeding, the latter often requiring packing of the nasal cavity. Subsequent removal of this packing can be quite painful. Also in many cases scar tissue may form in the nasal cavity, necessitating painful post-operative “debridements,” in which the ENT physician cuts out scar tissue in the physician's clinic, typically on an awake patient. Also, since the nasal and sinus tissue are significantly traumatized by a FESS procedure, it may take several days to weeks to know whether the surgery was successful.

FIG. 1 illustrates a frontal anatomical representation (parallel to the coronal plane) showing three of the four pairs of paranasal sinuses—frontal sinuses FS, ethmoid sinuses ES, maxillary sinuses MS—located within a patient's head H. The frontal sinuses FS are located above and behind the eyebrows. The ethmoid sinuses ES, which are formed as a “honeycomb” structure of several individual air cells, are located between the eyes. The maxillary sinuses MS are located behind the cheeks. The sphenoid sinuses, which are not shown in FIG. 1, are located posterior to the ethmoid sinuses.

FIG. 2 is another frontal view of the sinuses located within the skull bone SK. The nasal septum NS divides the nasal cavity into left and right sides. Because the following described structures are generally symmetrical bilaterally, only one of the paired structures is illustrated for sake of convenience. Within the nasal cavity are the middle turbinate MT and the inferior turbinate IT. The middle turbinate MT is connected to the base of the skull SK, while the inferior turbinate IT is connected to the lateral wall of the sinus cavity. The turbinates MT, IT have an underlying bony structure, but are covered with a lining of mucosal tissue (“mucosa lining ML”). When this lining swells (rhinitis), it can inhibit breathing through the nose, particularly the inferior turbinate IT. The ethmoid sinuses ES are depicted by a single air cell in FIG. 2. The uncinate process UP is a complex three-dimensional structure, projecting off of the lateral wall like a crescent-shaped leaf. The curved aspect of the medial bone defining the ethmoid sinuses ES is called the ethmoid bulla EB. The passageway between the ethmoid bulla EB and the uncinate process UP is referred to as the infundibulum I. The drainage path of the maxillary MS, frontal FS, and some of the ethmoid ES air cells runs into the infundibulum I. At the most inferior part of the maxillary sinus is a thin portion of skull bone referred to as the canine fossa CF. Though this is not a true opening, it is a relatively thin bone region, just above the root of the outer aspect of the canine teeth, inside the mouth. The relationship of the sinuses to the orbit O of the eye can also be seen. The sinus cavities have a mucosa lining ML disposed over the bone.

FIG. 3A is a side view parallel to the sagittal plane, looking at the right lateral nasal wall. The right nostril N is seen. The sphenoid sinus SS and frontal sinus FS may also be seen in this view. The flap-like structures illustrated in FIG. 3A are the inferior turbinate IT and middle turbinate MT. A portion of the lateral nasal wall under the middle turbinate MT is called the middle meatus MM, and a portion of the lateral wall under the inferior turbinate IT is called the inferior meatus IM. Other structures of the nasal cavity have been left out for clarification, e.g., the superior turbinate.

Referring to FIG. 3B, structures of the lateral nasal wall located underneath the middle turbinate MT are shown, with the middle turbinate MT lifted up. As seen in FIG. 3A, the ethmoid bulla EB is shown. Behind the wall of the ethmoid bulla EB are one or more of the individual air cells of the ethmoid sinus ES (not shown). Anterior and inferior to the ethmoid bulla, the uncinate process UP is shown having a free edge FE and a connected edge CE. The free edge FE stands out from the nasal wall, while the connected edge CE connects the structure to the nasal wall.

In FIG. 3C, the uncinate process UP has been cut away to reveal the underlying anatomy. Two ostia (naturally existing openings into paranasal sinuses) are visible in this view—the maxillary sinus ostium MO and the frontal sinus ostium FO. The frontal sinus FS and maxillary sinus MS drain mucus into the infundibulum I through their respective ostia. Drainage problems can arise in and/or extend from the ostia of one or more of these sinuses to the infundibulum I or vice versa. Consequently, conventional FESS surgical treatment of sinusitis typically involves widening one or more of the ostia FO, MO, as well as complete removal of the uncinate process UP. In fact, removal of the uncinate process UP is usually required even to just allow visualization of these sinus ostia FO, MO for the proper placement of the various surgical cutting instruments. FESS procedures also often include complete or partial removal of the ethmoid sinuses ES.

The foregoing description makes it readily apparent that the anatomy of the paranasal sinuses and the nasal cavity is very complex. The passageways through the nasal cavity and from the cavity to the sinuses are quite tortuous, and because the anatomy is made up of bone covered by mucosa, it can be quite challenging to navigate instruments through the anatomy without harming nasal/paranasal structures. It can also be challenging to treat chronic sinusitis, and it is sometimes advantageous to treat the condition using a combination of devices and/or a combination of procedures. Using multiple devices and/or performing multiple procedures in the tortuous anatomy of the nasal cavity and paranasal sinuses often presents challenges for an ENT surgeon. This is one of the reasons that alternative approaches and improved techniques are continually being researched.

More recently, others have suggested a method for treating a maxillary sinus by creating a hole into the sinus through an area in the mouth, at the top of the gums, called the canine fossa, passing a balloon catheter through the manmade hole, and dilating the maxillary sinus ostium with the balloon catheter. This method is described in further detail in U.S. patent application Ser. Nos. 11/399,691, 11/782,612, 11/782, 620, 11/782,617, 11/782,623, 11/782,624 and 11/623,740, the full disclosures of which are hereby incorporated by reference. However, there may be ways to improve upon the method described in the above-referenced patent applications. Thus, the present invention addresses various alternative approaches and improved methods, devices and systems for performing paranasal sinus procedures.

### SUMMARY

The embodiments described below are generally directed to alternative approaches to accessing and/or treating one or more paranasal sinuses, such as in treatment of sinusitis. Generally, improved methods for accessing a maxillary sinus via an opening formed in the canine fossa and procedures to perform once access is gained are described. Additionally, the description addresses various alternative access routes to the maxillary and other sinuses, as well as alternative balloon configurations for balloon dilation catheters.

In one aspect of the present invention, a method for dilating a maxillary paranasal sinus ostium in a patient may involve: forming an opening into a maxillary sinus through a canine fossa of a patient with a dual-lumen cannula; advancing a balloon catheter out of a distal opening in a first lumen of the cannula; viewing the balloon catheter using an endoscope disposed in a second lumen of the cannula; and expanding a balloon of the balloon catheter within an ostium of the maxillary sinus to dilate the ostium, causing breakage or rearrangement of bone underlying mucosa of the ostium, while viewing the expansion using the endoscope. In some embodiments, forming the opening may involve piercing through the canine fossa with a sharp distal tip of the cannula. Alternatively, forming the opening may involve piercing through the canine fossa with a sharp-tipped piercing device disposed within one of lumens of the cannula and removing or retracting the piercing device through the cannula. In one embodiment, the method may also involve deflating the balloon, advancing the balloon into a frontal sinus outflow tract or frontal sinus ostium, and expanding the balloon to dilate the frontal sinus outflow tract or frontal sinus ostium, causing breakage or rearrangement of bone underlying mucosa of the ostium.

In some embodiments, before advancing the balloon catheter, the method may also involve advancing a guide into the maxillary sinus, with the balloon catheter then being advanced over or through the guide. In one embodiment, for example, the guide may be a light emitting guidewire, and the method may further involve emitting light from the light emitting guidewire into the maxillary sinus and viewing the emitting light from outside the patient, through the patient's skin, to confirm positioning of the guidewire in the maxillary sinus.

Optionally, the method may also involve advancing a distal end of the endoscope out of a distal opening in the second lumen. In some embodiments, a proximal end of the endoscope may be coupled with the patient or an object outside the patient to secure the endoscope during the procedure, thus allowing for “hands free” visualization using the endoscope.

In one embodiment, the method may also include removing the balloon catheter from the maxillary sinus through the cannula, advancing an irrigation or suction catheter through the first lumen into the maxillary sinus, and irrigating or suctioning the maxillary sinus using the irrigation catheter. Optionally, the method may involve removing the balloon catheter from the maxillary sinus through the cannula, advancing a tissue removal device through the first lumen into the maxillary sinus, and removing tissue from the maxillary sinus using the tissue removal device. Any of a number of other procedures may be performed through the canine fossa opening, examples of which are described further below.

In another aspect of the present invention, a method for treating a paranasal sinus in a patient may include forming an opening through a canine fossa into a maxillary paranasal sinus and performing at least one procedure other than a balloon dilation procedure within the maxillary sinus or within another paranasal sinus of the patient's head, using a device advanced through the opening in the canine fossa. In some embodiments, the method may further involve advancing a guide through the opening in the canine fossa, with the device being advanced into the maxillary or other sinus over or through the guide.

Any of a number of different procedures other than a balloon dilation may be performed through the canine fossa opening. For example, in one embodiment, performing the procedure may involve injecting fluid through the canine fossa into the maxillary sinus and visualizing the maxillary sinus using an imaging method such as but not limited to ultrasound, computed tomography or magnetic resonance imaging. In some embodiments, performing the procedure may involve irrigating the maxillary sinus using a sinus irrigation catheter advanced through the canine fossa opening. Performing the procedure may also include suctioning substance from the maxillary sinus using a suction catheter advanced through the canine fossa opening. In one embodiment, performing the procedure may involve inflating a large balloon within the maxillary sinus to push mucus out of the sinus. In another example, performing the procedure may involve removing polyp tissue from inside the maxillary sinus using a tissue removal device advanced through the canine fossa opening. In yet another embodiment, performing the procedure may involve advancing a drug eluting sinus spacer through the canine fossa opening and into the maxillary sinus, a frontal sinus, a frontal sinus outflow tract, an ethmoid sinus or a sphenoid sinus and leaving the sinus spacer in place for a period of between 1 and 180 days.

Some methods may further involve placing a one-way valve in the canine fossa opening, where the procedure is performed through the valve. Optionally, a method may also include inserting a dilator device into the opening in the canine fossa and using the dilator to dilate or expand the opening. The method may further include using a nerve detection device to detect at least one nerve adjacent the canine fossa prior to forming the canine fossa opening. In some embodiments, the method may also include advancing an endoscope into the maxillary sinus through the canine fossa opening and visualizing at least one of the device or an inner wall of the maxillary sinus using the endoscope. In one embodiment, the method may also include changing a viewing angle of the endoscope by adjusting a swing prism in the endoscope. Any of the procedures described above may also be performed in combination during a given procedure on a patient or other human or animal subject. Procedures may be performed on any sinus or combination of sinuses and in any order or combination.

In some embodiments, the method may also involve performing a balloon dilation procedure. This procedure may include, for example, inserting a balloon catheter through the canine fossa opening into the maxillary sinus, advancing the balloon catheter to position a balloon of the catheter in a maxillary sinus ostium, and expanding the balloon to dilate the maxillary sinus ostium, causing breakage or rearrangement of bone underlying mucosa of the ostium. Optionally, the method may further include deflating the balloon, advancing the balloon catheter through the maxillary sinus ostium and into a frontal sinus outflow tract or frontal sinus ostium, expanding the balloon to dilate the frontal sinus outflow tract or frontal sinus ostium, causing breakage or rearrangement of bone underlying mucosa of the ostium, deflating the balloon, and removing the balloon catheter through the canine fossa opening.

In some embodiments, the method may further include, before advancing the balloon catheter, advancing a guide into the maxillary sinus through the canine fossa opening, where the balloon catheter is advanced over or through the guide. For example, in one embodiment, the guide may be an illuminating guidewire, and the method may further include emitting light from the guidewire at or near its distal end and viewing the emitted light from outside the patient to confirm positioning of the guidewire distal end in the maxillary sinus. In another embodiment, the guide may be a cannula, and the catheter may be advanced through a lumen of the cannula. In some embodiments, forming the opening through the canine fossa may involve piercing the canine fossa with a piercing member coupled with the cannula.

In another aspect of the present invention, a method for treating a maxillary paranasal sinus in a patient may involve: forming an opening through a canine fossa into a maxillary paranasal sinus; advancing a distal end of a guidewire through the opening and through a maxillary sinus ostium, into a nostril of the patient's head; advancing a balloon catheter into the nostril; coupling the balloon catheter with the distal end of the guidewire; advancing the balloon catheter into the maxillary sinus ostium using the guidewire; and expanding the balloon to dilate the maxillary sinus ostium. In some embodiments, coupling the balloon catheter with the distal end of the guidewire may involve attaching a first attachment member disposed on the catheter with a second attachment member disposed on the guidwire, where the catheter is advanced into the ostium by pulling the guidewire back through the ostium. Alternatively, coupling the balloon catheter with the distal end of the guidewire may involve advancing the catheter over the guidewire. In one embodiment, the method may involve viewing the balloon catheter in the nostril using an endoscope positioned in the nostril. In one embodiment, the method may further involve viewing a distal end of the balloon catheter in the maxillary sinus ostium using an endoscope positioned in the maxillary sinus via the canine fossa opening.

In another aspect of the present invention, a method for dilating a maxillary paranasal sinus ostium may include: forming an opening through an inferior meatus of a nasal cavity into a maxillary sinus; advancing a guide device into the nasal cavity to a position in or near the opening; advancing a flexible balloon catheter through or over the guide to position a balloon of the catheter in the maxillary sinus ostium; and expanding the balloon to dilate the maxillary sinus ostium, causing breakage or rearrangement of bone underlying mucosa of the ostium. In some embodiments, the opening may be formed anterior to a lacrimal duct in the nasal cavity.

In another aspect of the present invention, a method for dilating a maxillary paranasal sinus ostium may include forming an opening through a palate in a mouth into a maxillary sinus, advancing a balloon catheter through the opening to position a balloon of the catheter in the maxillary sinus ostium, and expanding the balloon to dilate the maxillary sinus ostium, causing breakage or rearrangement of bone underlying mucosa of the ostium. In one embodiment, the method may also include viewing at least part of the method using an endoscope advanced through a nostril of the patient. Optionally, the method may further involve forming a flap of mucosal tissue overlying bony tissue of the palate, before forming the opening and securing the flap of tissue back onto the palate after dilating the sinus ostium and removing the balloon catheter.

In another aspect of the present invention, a method for dilating a maxillary paranasal sinus ostium may involve: forming an opening through an uncinate process in a nasal cavity into a maxillary sinus; advancing a guide device into the nasal cavity to a position in or near the opening; advancing a balloon catheter through the opening to position a balloon of the catheter in the maxillary sinus ostium; and expanding the balloon to dilate the maxillary sinus ostium, causing breakage or rearrangement of bone underlying mucosa of the ostium. In some embodiments, the method may further include viewing at least part of the method using an endoscope advanced through a nostril of the patient.

In another aspect of the present invention, a method for dilating a maxillary paranasal sinus ostium may involve: forming an opening through a posterior fontanelle into a maxillary sinus; advancing a guide device into the nasal cavity to a position in or near the opening; advancing a flexible balloon catheter through the opening to position a balloon of the catheter in the maxillary sinus ostium; and expanding the balloon to dilate the maxillary sinus ostium, causing breakage or rearrangement of bone underlying mucosa of the ostium.

In another aspect of the present invention, a method for treating a maxillary paranasal sinus in a patient may involve: forming an opening through a canine fossa into a maxillary paranasal sinus; advancing a balloon catheter through the canine fossa opening to position a balloon of the catheter in a maxillary sinus ostium; expanding the balloon to dilate the maxillary sinus ostium; removing the balloon catheter from the patient; and placing a tissue dressing over the opening in the canine fossa. In some embodiments, the tissue dressing may include an adhesive layer and a barrier layer. Optionally, the adhesive layer, the barrier layer or both may include a therapeutic substance.

In another aspect of the present invention, a system for treating a maxillary paranasal sinus may include: a cannula for forming an opening through a canine fossa into a maxillary paranasal sinus, wherein the cannula includes at least two lumens for guiding at least two devices into the maxillary sinus; a balloon catheter configured to be inserted through a first lumen of the cannula; and an endoscope configured to be inserted through a second lumen of the cannula. In one embodiment, the cannula may include a sharp tip, and the system may further include at least one lumen filling member disposed within the lumens during advancement of the cannula through the canine fossa. In an alternative embodiment, the cannula may have a blunt tip, and the system may further include at least one sharp-tipped device removably disposed within at least one of the lumens for forming the opening through the canine fossa.

In some embodiments, the system may further include at least one additional device insertable through at least one of the lumens of the cannula after removal of the balloon catheter or the endoscope. For example, the additional device may be, but is not limited to, a tissue removal tool, a suction catheter, an irrigation catheter, a combined suction/irrigation catheter, an illuminating guidewire, a non-illuminating guidewire and/or a drug eluting sinus spacer. In some embodiments, the system may also include a mucosal tissue dressing for applying over the canine fossa opening after the cannula is removed. In some embodiments, the cannula may have an angled portion configured to direct the balloon catheter toward an ostium of the maxillary paranasal sinus. In some embodiments, the balloon catheter may have a length sufficient to extend from outside the patient, through a maxillary sinus ostium, and to a frontal sinus outflow tract or frontal sinus ostium.

In another aspect of the present invention, a system for treating a maxillary paranasal sinus may include a piercing device for piercing an opening through a canine fossa into a maxillary paranasal sinus, a one-way valve configured for placement in the opening, and a balloon catheter configured to be inserted through the one-way valve. In some embodiments, the system may further include at least one additional device insertable through the one-way valve, such as but not limited to an endoscope, a tissue removal tool, a suction catheter, an irrigation catheter, a combined suction/irrigation catheter, an illuminating guidewire, a non-illuminating guidewire and a drug-eluting sinus spacer.

In another aspect of the present invention, a system for treating a paranasal sinus may include: a sheath; a trocar insertable through the sheath and including a point for piercing a canine fossa; a cannula for inserting through the sheath; a balloon catheter configured to be inserted through the cannula; a flexible endoscope configured to be inserted through the cannula; and at least one additional device insertable through the cannula. For example, the additional device may include, but is not limited to, a tissue removal tool, a suction catheter, an irrigation catheter, a combined suction/irrigation catheter, an illuminating guidewire, a non-illuminating guidewire and/or a drug-eluting sinus spacer. In some embodiments, the system may further include one or more mucosal tissue dressings for covering the opening in the canine fossa after completion of a procedure.

Further aspects and embodiments are described in further detail below in reference to the attached drawings.

### BRIEF DESCRIPTION OF THE DRAWINGS

FIG. 1 is a schematic view illustrating the paranasal sinuses in relation to the face;

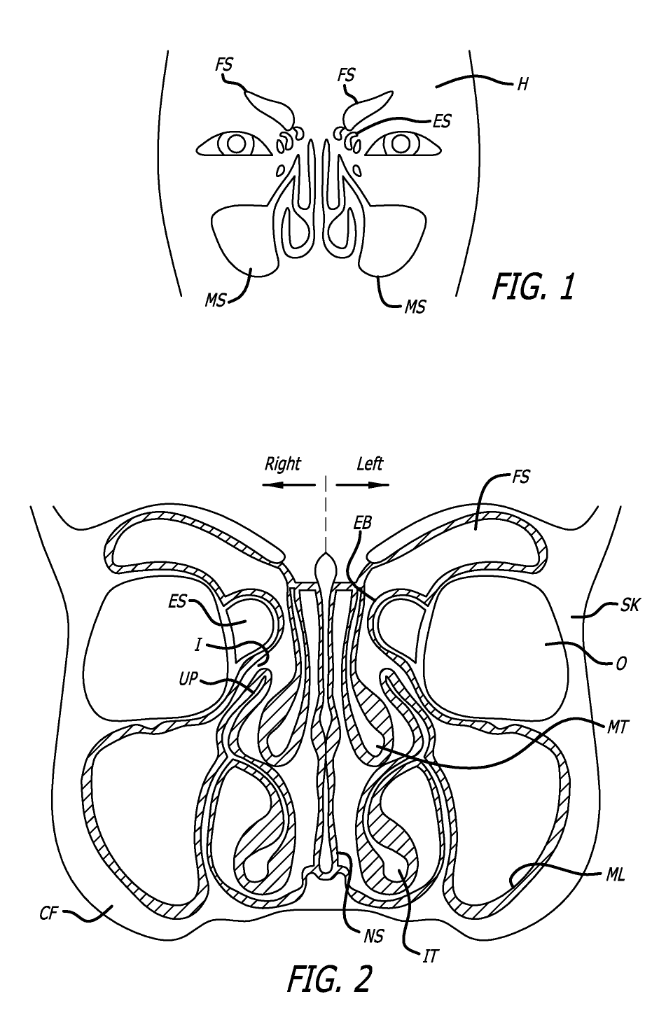

FIG. 2 is a coronal section of the human skull, showing the paranasal sinuses;

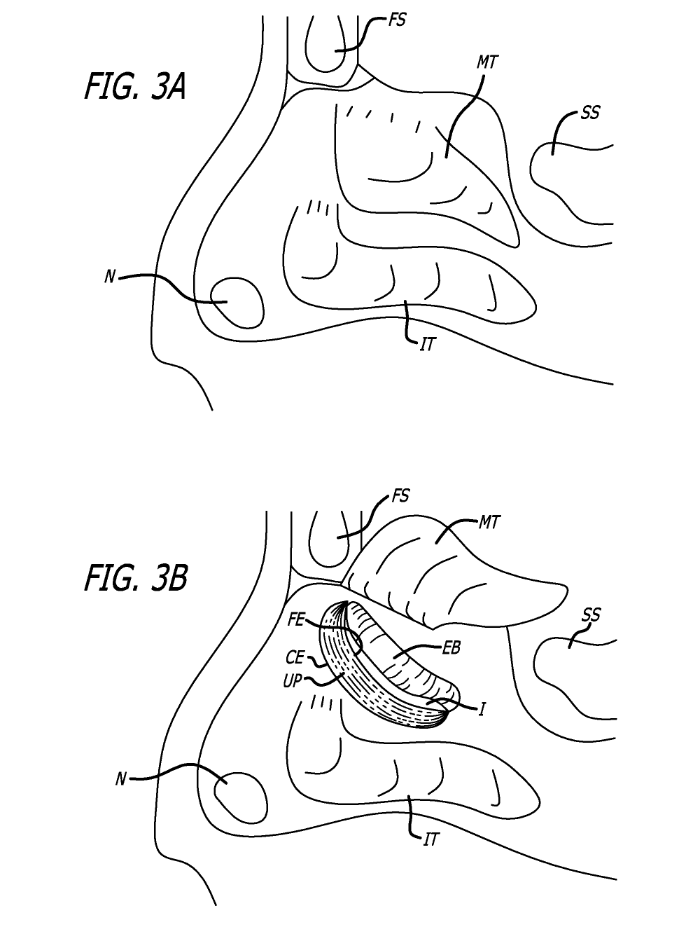

FIGS. 3A-3C are sagittal views of the lateral nasal wall, illustrating various anatomical features;

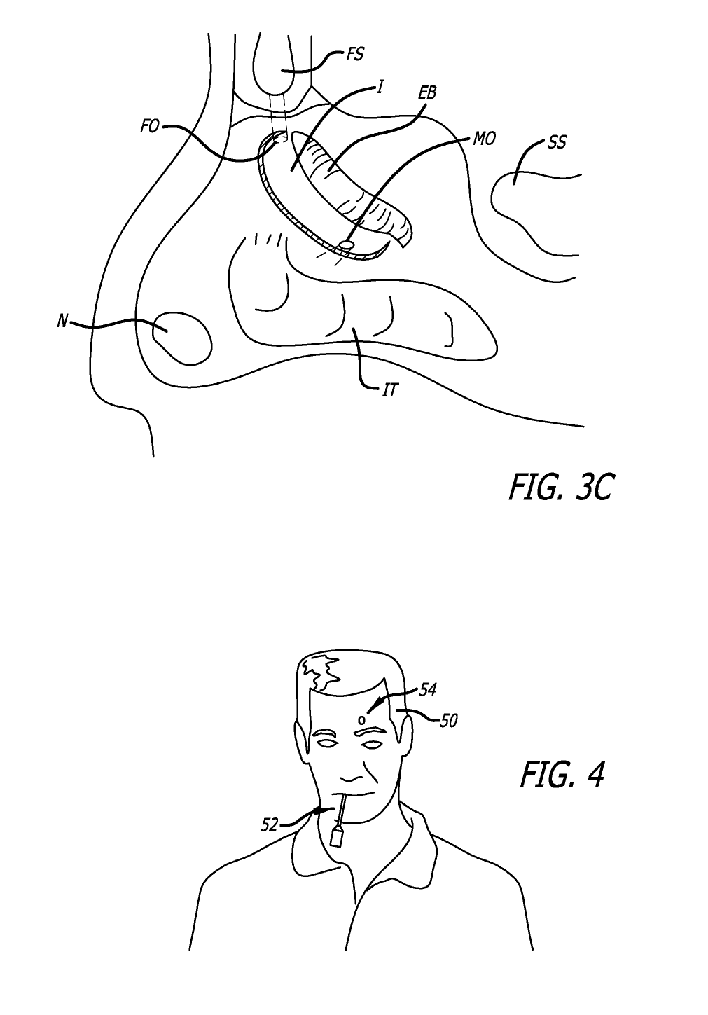

FIG. 4 is a front view of a patient's head, depicting access approaches to a maxillary sinus and a frontal sinus;

FIGS. 5A and 5B are cross-sectional views of a maxillary sinus and nasal cavity, demonstrating a method for accessing a maxillary sinus and dilating the maxillary sinus ostium, according to one embodiment of the present invention;

FIG. 5C is a cross-sectional views of the cannula of FIGS. 5A and 5B;

FIG. 5D is a cross-sectional view of a cannula, according to an alternative embodiment of the present invention;

FIG. 6 is a cross-sectional view of a maxillary paranasal sinus showing a canine fossa access sheath and balloon catheter, according to one embodiment of the present invention;

FIGS. 7A and 7B are cross-sectional views of a maxillary paranasal sinus showing a canine fossa access sheath and close-up view of the sheath including a one-way valve, according to one embodiment of the present invention;

FIG. 7C is a cross-sectional view of a maxillary paranasal sinus showing a method for balloon dilating a manmade canine fossa opening, according to one embodiment of the present invention;

FIG. 8 is a cross-sectional view of a maxillary paranasal sinus showing a canine fossa access cannula and illuminating guidewire, according to one embodiment of the present invention;

FIGS. 9A and 9B are cross-sectional views of a maxillary paranasal sinus showing a canine fossa access cannula and method for advancing an illuminating guidewire and balloon catheter through a maxillary sinus ostium to a frontal sinus ostium, according to one embodiment of the present invention;

FIG. 10 is a cross-sectional view of a maxillary paranasal sinus showing a canine fossa access cannula and an endoscope with a balloon protected tip, according to one embodiment of the present invention;

FIGS. 11 and 12 are partial cross-sectional views of distal ends of swing prism endoscopes, according to alternative embodiments of the present invention;

FIGS. 13A and 13B are cross-sectional views of a maxillary paranasal sinus demonstrating a method for using a transnasally inserted swing prism scope to view a balloon dilation procedure, according to one embodiment of the present invention;

FIG. 14A is a cross-sectional view of a maxillary paranasal sinus showing a canine fossa access cannula and an irrigation catheter advanced into the sinus, according to one embodiment of the present invention;

FIG. 14B is a cross-sectional view of a maxillary paranasal sinus showing a canine fossa access cannula with suction and irrigation capabilities, according to one embodiment of the present invention;

FIG. 15 is a cross-sectional view of a maxillary paranasal sinus showing a canine fossa access cannula and a tissue removal device in the sinus, according to one embodiment of the present invention;

FIG. 16 is a cross-sectional view of a maxillary paranasal sinus showing a canine fossa access cannula and a drug eluting sinus spacer in the sinus, according to one embodiment of the present invention;

FIG. 17 is a cross-sectional view of a maxillary paranasal sinus showing a needle advanced into the sinus for injecting and/or removing fluid, according to one embodiment of the present invention;

FIG. 18 is a cross-sectional view of a maxillary paranasal sinus showing a canine fossa access sheath and a space occupying balloon inflated in the sinus to push fluid out of the sinus, according to one embodiment of the present invention;

FIGS. 19A and 19B are cross-sectional views of a maxillary paranasal sinus showing a canine fossa access cannula and a procedure for advancing a balloon catheter into a maxillary sinus ostium over a guidewire, according to one embodiment of the present invention;

FIGS. 20A and 20B illustrate a mucosal tissue dressing for placement over a puncture site into a maxillary sinus or other sinus, according to one embodiment of the present invention;

FIG. 21 is a cross-sectional coronal view of the anterior portion of a skull, showing an alternative access approach to a maxillary paranasal sinus, according to one embodiment of the present invention;

FIGS. 22A and 22B are cross-sectional side views of a lateral nasal wall, showing an alternative access approach to a maxillary paranasal sinus, according to one embodiment of the present invention;

FIG. 23 is a cross-sectional view of a maxillary sinus, showing an alternative access approach to a maxillary paranasal sinus, according to one embodiment of the present invention;

FIG. 24 is a cross-sectional coronal view of the anterior portion of a skull, showing an alternative access approach to a maxillary paranasal sinus, according to one embodiment of the present invention;

FIG. 25 is a cross-sectional side view of a lateral nasal wall, showing an alternative access approach to a maxillary paranasal sinus, according to one embodiment of the present invention; and

FIGS. 26A and 26B are perspective views of alternative embodiments of a distal portion of a balloon catheter, according to alternative embodiments of the present invention.

### DETAILED DESCRIPTION

### Improved Canine Fossa Access to the Maxillary Sinus

The canine fossa CF is a thin portion of the maxillary sinus wall located adjacent the root of the canine teeth. For the purposes of this application, the canine fossa CF refers to the general region or anatomical area of, surrounding and/or adjacent to the canine fossa CF and is not limited to a single, discrete structure or location. Forming an opening through the canine fossa CF to access a maxillary sinus is a technique that has been used in the past and that is described in U.S. patent application Ser. Nos. 11/399,691, 11/782,612, 11/782, 620, 11/782,617, 11/782,623, 11/782,624 and 11/623,740, which were previously incorporated by reference. The following embodiments provide improvements to a canine fossa CF access approach to maxillary paranasal sinuses.

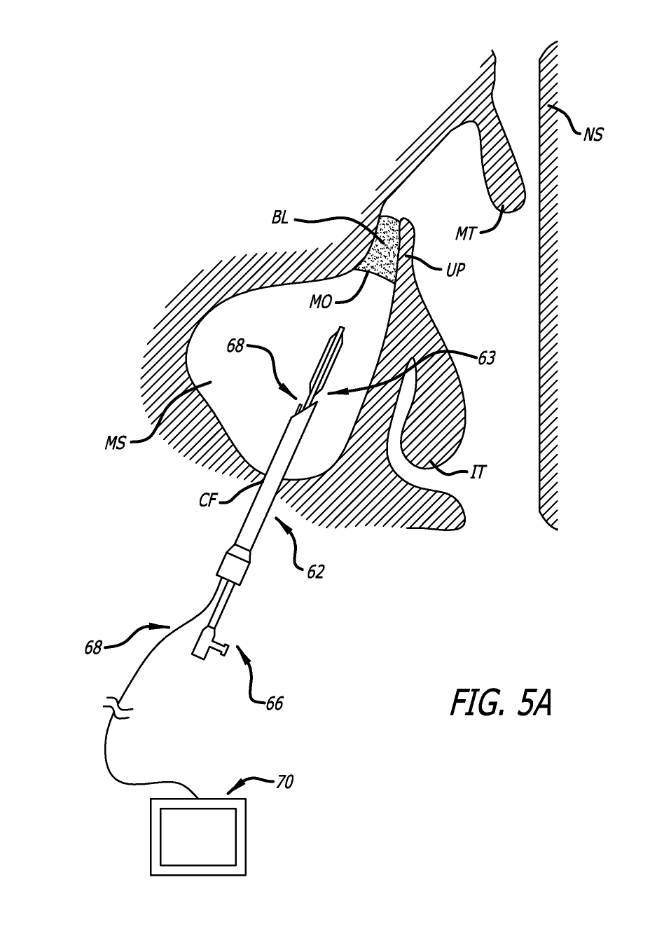

Turning to FIG. 4, in one embodiment, a maxillary sinus of a patient 50 may be accessed by forming an opening through a canine fossa CF using a cannula 52. In some embodiments, a frontal sinus FS may also (or alternatively) be accessed directly from outside the skull, through the wall of the frontal sinus FS to facilitate treatment of the frontal sinus ostium FO. In some embodiments, rather than using a trocar, a frontal sinus FS may be directly accessed through a mini-trephination through the skin and the sinus wall, using a drill tool to form the opening into the sinus. Once accessed, any suitable procedure may be performed on a frontal sinus FS, many examples of which are described further below in relation to the maxillary sinuses. For example, a balloon catheter may be advanced directly into a frontal sinus ostium FO, either alone or over a guidewire, and used to dilate the ostium. In some cases, visualization may not be required, while in other cases visualization may be acquired using, for example, an endoscope advanced through the nostril.

In alternative embodiments, some of which are described in greater detail below, initial access to a first sinus may be used to access a second sinus. For example, access to a maxillary sinus through an opening formed in the canine fossa may be used to access an ethmoid, frontal and/or sphenoid sinus. In one embodiment, described in further detail below, a working tool may be advanced through the opening in the canine fossa and then through the maxillary sinus ostium to access an ethmoid, frontal and/or sphenoid sinus. In various embodiments, any combination or number of sinuses may be accessed and treated. Therefore, while the description below primarily addresses the canine fossa access approach and/or a transnasal (through the nostril) approach to the maxillary sinus, most if not all embodiments may alternatively be used through a direct opening into a frontal sinus and/or in any paranasal sinus or combination of sinuses through the canine fossa access approach.

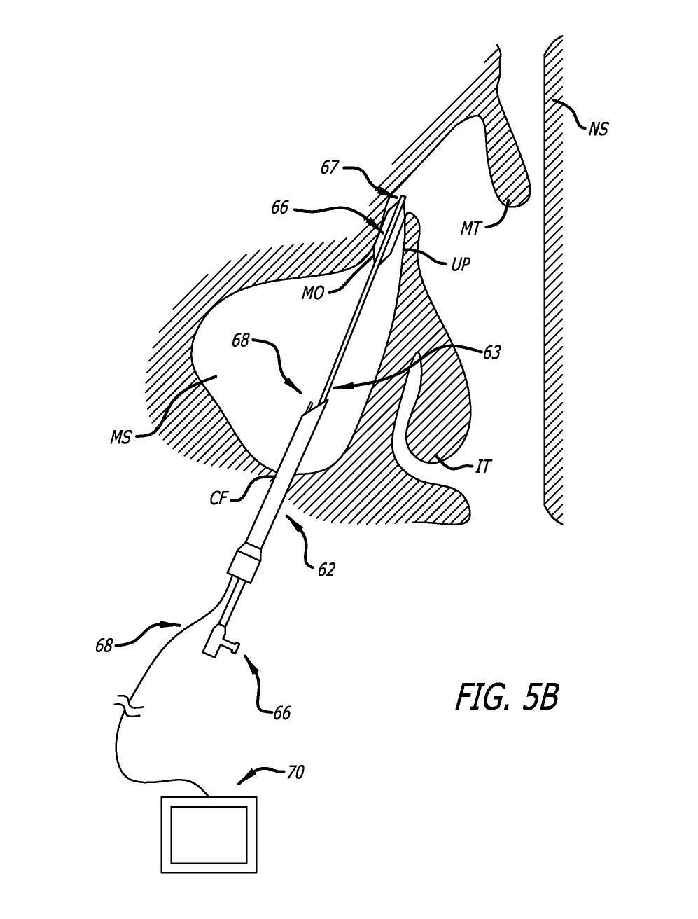

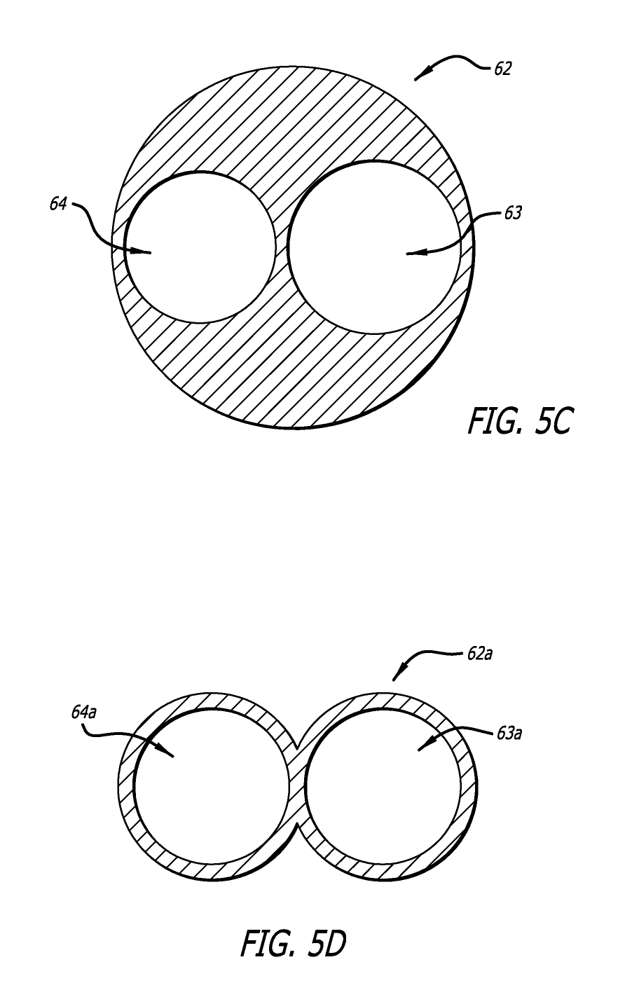

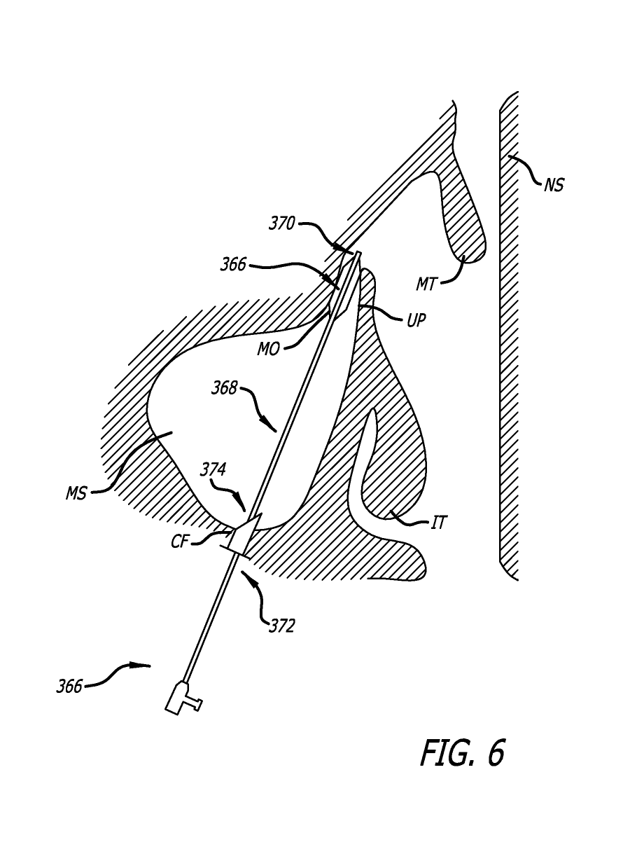

Referring now to FIGS. 5A-5D, one embodiment of a method for accessing and treating a maxillary sinus MS is illustrated. First, a multi-lumen cannula 62 having a sharp distal tip 63 may be advanced through the canine fossa CF of a patient and into the maxillary sinus MS. Providing direct access to the maxillary sinus MS with cannula 62 avoids the necessity of a separate piercing device, such as a trocar, and a cannula or sheath device for passage of instruments, since cannula 62 achieves both of these functions. In some embodiments, cannula 62 includes sharp distal tip 63, as shown, and may also include a removable inner stylet (not shown), which may be removed after cannula 62 is advanced into a maxillary sinus MS. In an alternative embodiment, cannula 62 may have a blunt distal tip rather than sharp tip 63, and in this case a sharp trocar may be positioned in a lumen of cannula 62 for piercing through the canine fossa CF and may subsequently be removed. Thus, in any of the embodiments shown in subsequent figures with a sharp tip 63, in alternative embodiments a dull tip/inner trocar combination may be substituted. After placement, any of a number of various medical devices may be advanced through cannula 62 to perform one or more procedures within the maxillary sinus MS, in the maxillary sinus ostium MO and/or in other sinuses, ostia or nasal cavity structures. Examples of such devices and procedures are described more fully below.

As shown in FIG. 5C, cannula 62 includes two lumens 63, 64 and may in fact include more lumens in alternative embodiments. Thus, referring again to FIG. 5A, in this embodiment a balloon dilation catheter 66 may be advanced through one lumen of cannula 62, and a flexible endoscope 68 (coupled with a viewing monitor 70) may be advanced through another lumen. Endoscope 68 may be any suitable endoscopic device, such as but not limited to a fiber optic scope, CCD scope, or the like. In alternative embodiments, cannula 62 may be large enough to accommodate a rigid endoscope in its endoscope lumen. In one embodiment, endoscope 68 is advanced through cannula 62 far enough so that its distal end reaches or passes just out of a distal end of cannula 62. Endoscope 68 may then be used to view the internal anatomy of the maxillary sinus, such as the maxillary sinus ostium MO, and/or balloon catheter 66 as it is advanced out of cannula 62, positioned in the ostium MO and used to dilate the ostium MO to relieve the blockage BL.

FIG. 5A shows balloon catheter 66 in its deflated state as it has just been advanced out of cannula 62. FIG. 5B shows balloon catheter 66 in it expanded state within the maxillary ostium MO. In some embodiments, catheter 66 may include an atraumatic distal tip 67. Also in some embodiments (not shown here), distal tip 67 may be angled relative to the longitudinal axis of catheter 66, which may facilitate steering of the distal end of catheter 66 toward the maxillary ostium MO. In various embodiments, any suitable balloon catheter may be used, such as but not limited to the Relieva Solo™ Sinus Balloon Catheters (Acclarent, Inc., Menlo Park, Calif.). In alternative embodiments, other, non-balloon expandable devices may be used to dilate a paranasal sinus ostium, such as but not limited to those described in U.S. patent application Ser. No. 11/789,705, entitled Mechanical Dilation of the Ostia of Paranasal Sinuses and Other Passageways of the Ear, Nose and Throat, and filed Apr. 24, 2007, the full disclosure of which is hereby incorporated by reference. The inflation device used to inflate balloon catheter 66 is not depicted in FIG. 5A, but any suitable inflation device may be used, such as but not limited to the Relieva® Sinus Balloon Inflation Device (Acclarent, Inc., Menlo Park, Calif.). Again, in these or other embodiments, once access is gained to the maxillary sinus MS, other procedures may be performed in addition to or as an alternative to balloon dilation of the ostium. Examples of such procedures are described more fully below.

FIG. 5C illustrates cannula 62 in cross section. In this embodiment, cannula 62 includes two lumens, a larger balloon catheter lumen 63 and a smaller flexible endoscope lumen 64. In alternative embodiments, any size, shape and number of lumens may be included. For example, various alternative embodiments of cannula 62 may include one or more irrigation lumens, suction lumens or combined suction/irrigation lumens. One embodiment may include a separate guidewire lumen, although typically a guidewire, when used, will be placed through the balloon catheter lumen and the balloon catheter will then be advanced over the guidewire.

FIG. 5D illustrates an alternative embodiment of a cannula 62a. This embodiment includes side-by-side balloon catheter 63a and endoscope 64a lumens with a smaller overall profile. In its various embodiments, cannula 62, 62a may have any suitable diameter, for example in the range of between about 1.0 mm and about 5.0 mm, and more preferably between about 1.5 mm and about 3.0 mm. Optionally, this or any other embodiment of a cannula 62, 62a may include one or more fastening members at or near its proximal end for temporarily fastening the cannula to a patient during a procedure, thus freeing up a hand of the physician user. Such fastening members may include, for example, a suture loop, a stabilizing member for resting against the patient's face, a strip of adhesive or the like.

In the embodiment shown in FIGS. 5A and 5B, cannula 62 has a straight configuration and is directed from the opening in the canine fossa CF toward the maxillary sinus ostium MO. In an alternative embodiment, a cannula (not shown) may include a preformed bend somewhere along its length in order to better aim the distal end of the cannula toward the ostium. Such a bend may facilitate directing a balloon catheter and endoscope passing through the cannula toward the ostium.

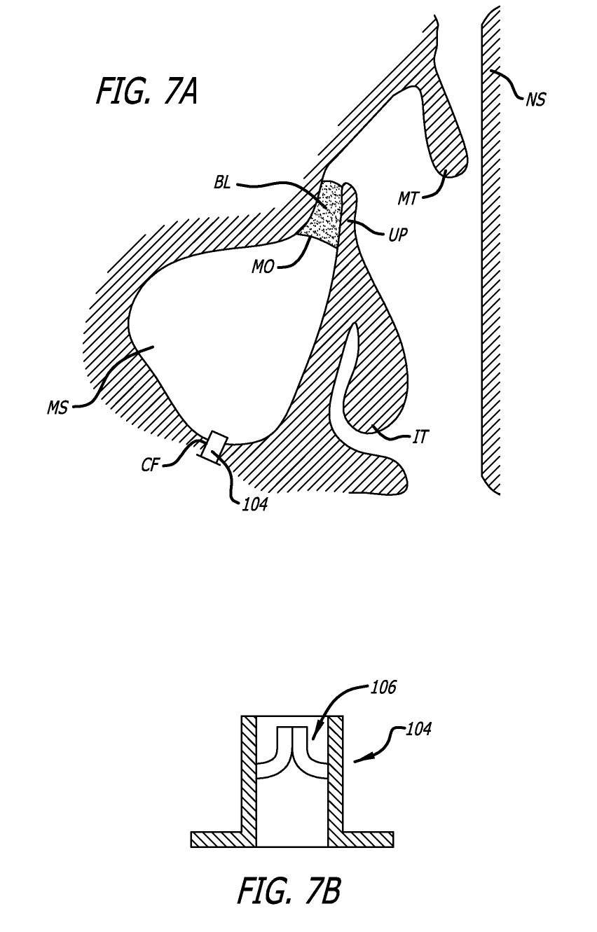

With reference now to FIG. 6, in another alternative embodiment, a generally tubular canine fossa access sheath 372 may be placed into an opening created through the canine fossa CF into the maxillary sinus MS. In the embodiment shown, sheath 372 includes a sharp distal end 374 so that it may be used to create the hole through the canine fossa CF. In an alternative embodiment, a piercing device, such as a trocar (not shown), may be used to form the opening through the canine fossa CF and sheath 372 may be positioned over the trocar, such that when the trocar is removed it leaves sheath 372 in place within the manmade opening. Once in place, access sheath 372 may act as a port through which one or more devices may be advanced into the maxillary sinus MS, such as a balloon catheter 366 with a rigid shaft 368 for dilating the maxillary sinus ostium MO. As with cannula 62, sheath 372 may include a fastening device in one embodiment for fastening to the gum, for example. In many embodiments, however, sheath 372 may remain in place in the canine fossa CF by the anatomy holding it in place.

In one embodiment, balloon catheter 366 with an at least partially rigid shaft 368 may be used with sheath 372. This embodiment allows the relatively short sheath 372 to be used for access, since the rigidity of shaft 368 allows catheter 366 to be advanced into the maxillary sinus MS and to the maxillary ostium MO without using a longer cannula or other guide device. Any rigid or semi-rigid balloon catheter 366 may be used, according to various embodiments. For example, in some cases Relieva Solo™ or Relieva Solo™ Pro Sinus Balloon Catheters (Acclarent, Inc., Menlo Park, Calif.) may be used. These catheters generally include a stiffer proximal portion and a more flexible distal portion. More rigid balloon catheters and methods for their use in dilating paranasal sinus ostia are described in greater detail in U.S. patent application Ser. Nos. 10/259,300, 11/347,147, 12/117,582, 12/117,672, 12/117,961, 12/118,931 and 12/120, 902, the disclosures of which are hereby incorporated fully by reference. By using a rigid endoscope in this embodiment along with a sheath, it is not necessary to use a cannula or other guide device to guide the balloon catheter toward the maxillary sinus ostium MO.

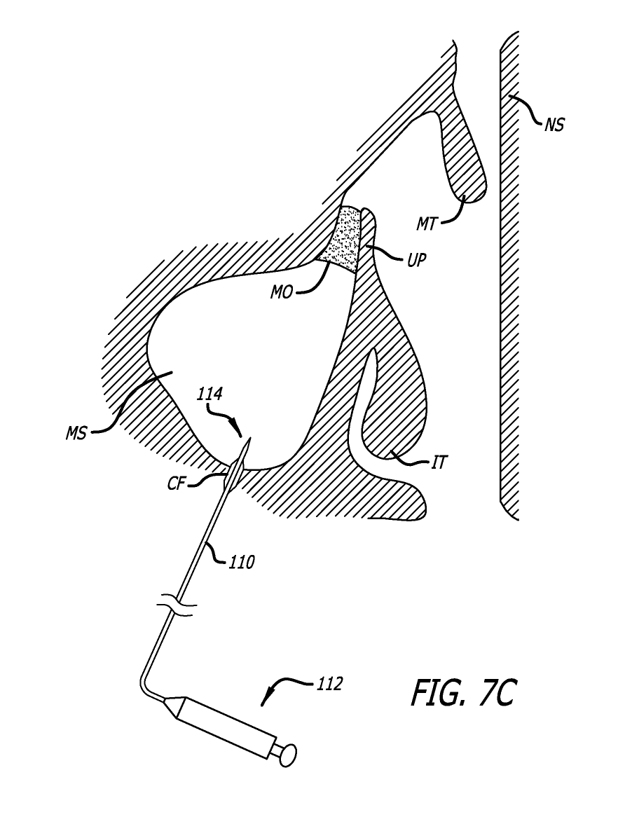

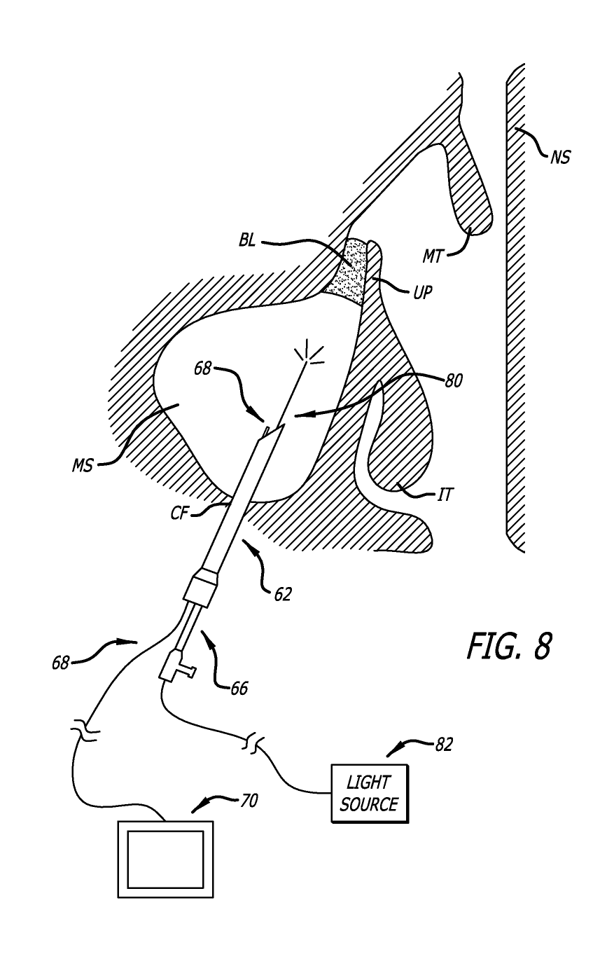

In one embodiment, and with reference now to FIGS. 7A and 7B, a canine fossa access sheath 104 may include a one-way valve 106. Valve 106 may allow devices to be passed in and out of the maxillary sinus through sheath 104 while preventing passage of fluids, such saliva or other oral cavity fluid, into the maxillary sinus MS. Valve 106 may thus help prevent sinus infections from oral cavity fluid passing into the sinus. In various embodiments, valve 106 may have any suitable construction and configuration. For example, in one embodiment, one-way valve 106 may include an internal bore configured with a duck-bill seal which functions to seal the opening, provide ingress to the sinus cavity and prevent egress from the cavity.

In some embodiments, sheath 104 may be left in place after a sinus procedure so that one or more later interventions may be performed through sheath, without requiring multiple canine fossa CF punctures. For example, one-way valve 106 may be accessed to inject or spray medication into the sinus, to perform an irrigation/lavage procedure, to remove one or more polyps, to biopsy tissue, to perform a diagnostic procedure or visualize the sinus with a visualization device, to perform an additional balloon dilation and/or the like.

Referring now to FIG. 7C, any of the above-described methods for accessing a maxillary sinus through a canine fossa puncture may, in some embodiments, be further enhanced by one or both of two additional techniques. First, a nerve detection device (not shown), such as a radiofrequency nerve stimulator or the like, may be used to detect nerves in and around the canine fossa before piercing through it to form the hole. It has been reported that a frequent side effect of piercing the canine fossa is numbness of the gum or teeth due to nerve damage caused by the piercing. Thus, locating nerves before piercing may help reduce that side effect. Second, in some cases it may be advantageous to dilate an opening into the canine fossa after the initial opening is formed, as shown in FIG. 7C. In one embodiment, a piercing device may be used to form the hole and then a balloon catheter 110 may be advanced into the hole and expanded via an inflation device 112 to widen the hole. In another embodiment, balloon catheter 110 may include a sharp distal tip 114 or needle and may thus be used to form the initial hole and to widen the hole. In some embodiments, a sheath, cannula or other access device may then be placed in the widened maxillary sinus access hole. The wider hole may also provide a drainage site for mucus and other fluids after the procedure.

Procedures in the Maxillary Sinus and/or Other Sinuses Via Canine Fossa Access

Once access is gained to a maxillary sinus via a canine fossa access approach, any of a number of procedures may be performed. Thus far, this description has focused on the example of a balloon dilation procedure for dilating a maxillary sinus ostium. In various embodiments, such a balloon dilation procedure may be combined with one or more additional procedures, or alternatively one or more procedures other than balloon dilation may be performed by themselves from within the maxillary sinus. Thus, the foregoing description focuses on various procedures that may be performed once sinus access is gained. In any given embodiment, canine fossa access to a maxillary sinus may be gained using any of the methods or devices described above or any of the methods or devices described in described in U.S. patent application Ser. Nos. 11/399,691, 11/782,612, 11/782, 620, 11/782,617, 11/782,623, 11/782,624 and 11/623,740, which were previously incorporated by reference. Additionally, as mentioned above, a similar array of procedures may be performed on a frontal sinus after access is gained via an opening into that sinus through the forehead. Additional access routes to the maxillary sinuses are described in greater detail below, and any of those alternative access routes may be used. Finally, access to one sinus may be gained by first accessing a different sinus and then advancing a device to the second sinus. At least one example of this method will be described in further detail below.

Any of the following procedures may be performed on any given sinus or sinuses, in any given order or combination, according to various embodiments. For example, many of the procedures may be performed as part of a balloon dilation procedure, either before or after a paranasal sinus ostium is dilated. In some embodiments, multiple procedures may be performed during the same operation on a patient. Alternatively, as described above, in some embodiments, a canine fossa access sheath may be left in place so that multiple procedures can be performed through the sheath over time. Thus, although each different procedure is described separately below, in use the procedures may be combined in any desired way to treat one or more sinuses.

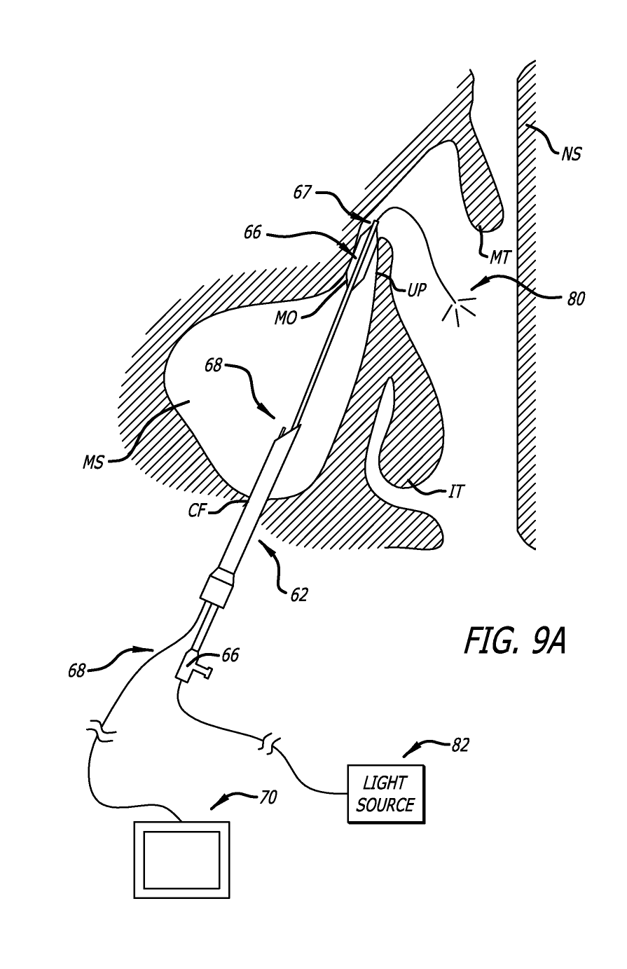

With reference now to FIG. 8, in one embodiment, a cannula 62 (or alternatively any other access device) may be used to access the maxillary sinus MS through the canine fossa CF, as was described in reference to FIG. 5A. An illuminating guidewire 80 may be advanced through cannula 62 to position its distal end in the maxillary sinus MS, and then light may be emitted from guidewire 80 as provided by a light source 82. The light from guidewire 80 may be viewed through the patient's skin (“sinus transillumination”), by the ENT physician, to confirm the distal end of guidewire 80 is in the sinus in a desired location. Subsequently, a balloon catheter 66 may be advanced over guidewire 80 and into the sinus ostium to perform a balloon dilation. In some embodiments, guidewire 80 may first be advanced through the ostium to act as a guide for balloon catheter 66. Also in some embodiments, a flexible endoscope 68 may be used during part or all of this method for visualization. Alternatively, since illuminating guidewire 80 helps visualize and confirm the location of itself and possibly balloon catheter 66, in some embodiments endoscope 68 may not be used.

Illuminating guidewire 80 may be, in one embodiment, the Relieva Luma™ Sinus Illumination Guidewire and System (Acclarent, Inc., Menlo Park, Calif.). Illuminating guidewires and methods for their use in paranasal sinuses are described in greater detail, for example, in U.S. patent application Ser. Nos. 11/522,497, entitled Methods and Devices for Facilitating Visualization in a Surgical Environment, and filed Sep. 15, 2006, and 12/122,884, entitled Sinus Illumination Guidewire Device, and filed May 19, 2008, the full disclosures of which are hereby incorporated by reference.

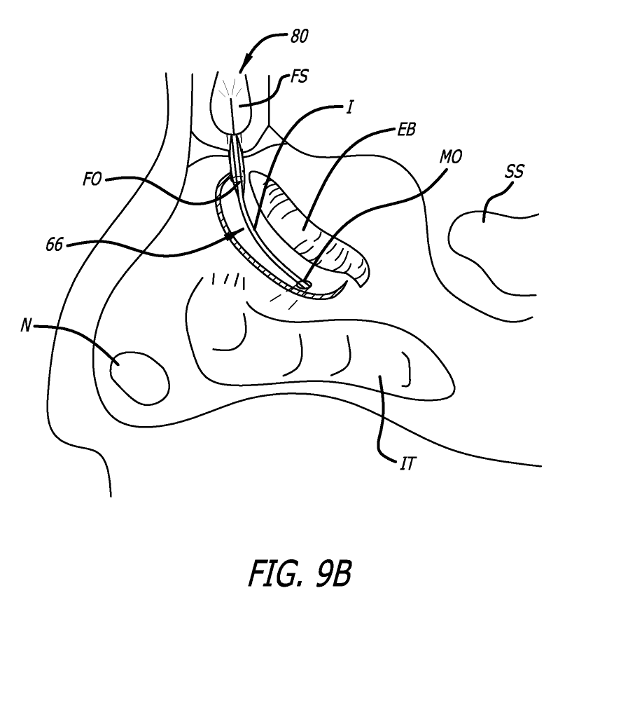

In some embodiments, and with reference now to FIGS. 9A and 9B, during or after balloon dilation of the maxillary sinus ostium, illuminating guidewire 80 may be advanced through the maxillary sinus ostium to illuminate other areas of the nasal and/or paranasal anatomy and/or to act as a guide for a balloon catheter to advance through the ostium to other locations. Alternatively, in some embodiments a non-illuminating guidewire may be used for this method. In various embodiments, guidewire 80 may be advanced while balloon catheter 66 is still expanded, as in FIG. 9. Alternatively, balloon catheter 66 may first be deflated and then guidewire 80 may be advanced.

Using this method, for example, and with reference to FIG. 9B, guidewire 80 may be directed into the frontal sinus outflow tract, the frontal sinus ostium and/or the frontal sinus itself. Progress of the illuminating guidewire 80 may be watched from outside the patient as an illumination spot moves from the maxillary ostium to the frontal sinus. Balloon catheter 66 may then be advanced over guidewire 80 to dilate the outflow tract and/or the ostium. In one embodiment, before advancing guidewire 80 and/or balloon catheter 66 further, cannula 62 may be advanced up to or through the maxillary sinus ostium MO to provide support and guidance. In such an embodiment, a longer version of cannula 62 may be provided and/or cannula 62 may have a blunt tip and/or an angled tip to prevent harming anatomical structures and to help guide guidewire 80 and balloon catheter 66 toward the frontal sinus. In alternative embodiments of this method, the maxillary sinus ostium may be dilated and subsequently the ethmoid sinus and/or the sphenoid sinus may be treated. In fact, any combination of sinuses may be treated sequentially in this manner.

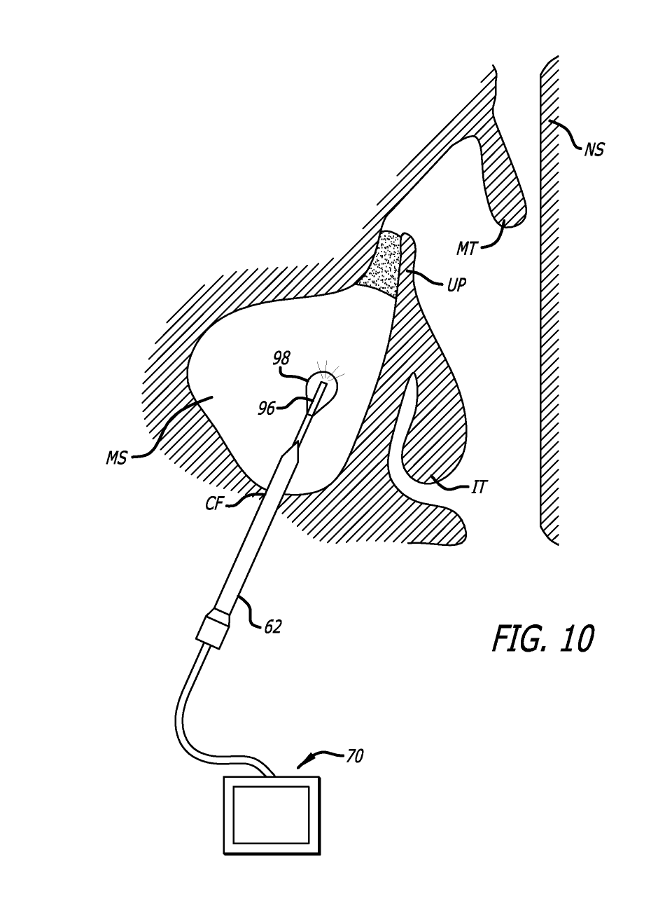

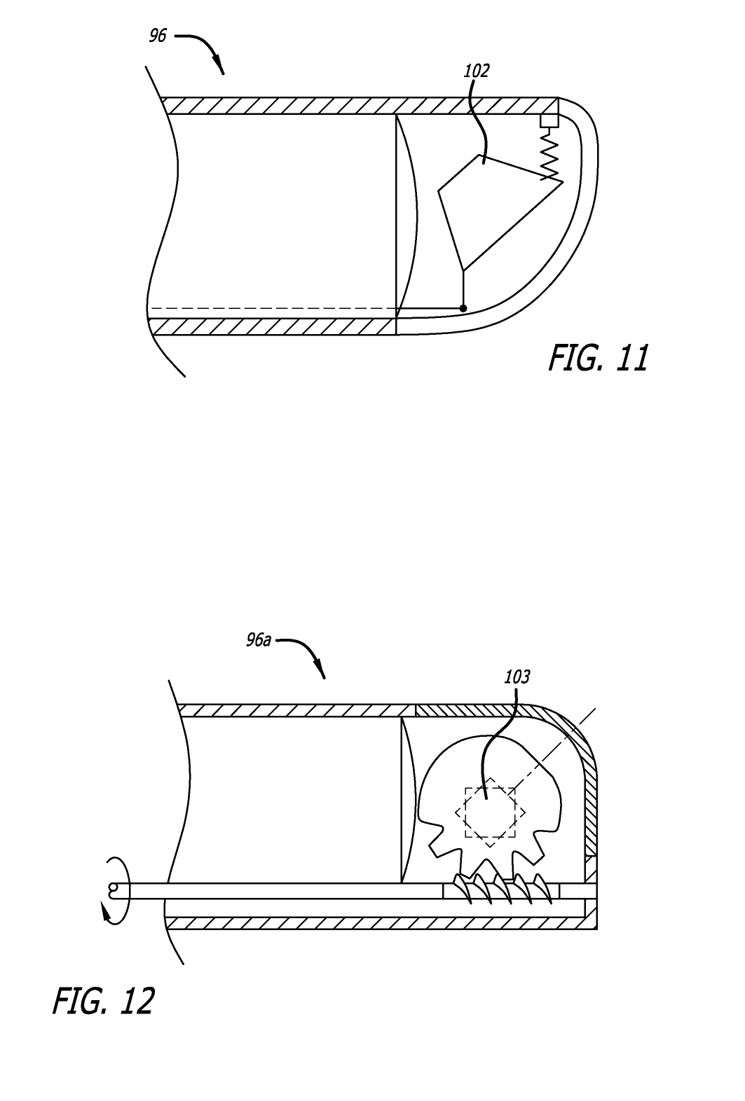

Referring now to FIGS. 10-12, in some embodiments rather than using a flexible endoscope, a rigid endoscope 96 may be inserted through a canine fossa piercing cannla 62 or other access device. In one embodiment, for example, endoscope 96 may have a protective balloon 98 disposed about the lens at its distal end, which may serve to keep the lens clean and to prevent any harm from endoscope 96 contacting the inner walls of the sinus. In an alternative embodiment, the tip of endoscope 96 may be incorporated into a balloon of a balloon catheter for the same purposes. Any type of endoscope 96 may be combined with balloon 98, but in one embodiment a swing prism endoscope 96 may be used. A shown in FIGS. 11 and 12, swing prism endoscopes 96, 96a include one or more mirrors or similar swing prism structures 102, 103 in or around their distal ends that are moveable to allow a user to change the viewing angle of the scope by moving the swing prism structure. Thus, a user may scan across multiple views without needing to move the body of the endoscope. Swing prism endoscopes for use in the nasal cavity and paranasal sinuses are described in greater detail in U.S. Provisional Patent Application Ser. No. 61/084,949, entitled Swing Prism Endoscope, and filed Jun. 30, 2008, the disclosure of which is fully incorporated herein by reference. In various embodiments, swing prism endoscope 96 or other rigid endoscopes may be used before a procedure to gain a better understanding of the anatomy or during a procedure.

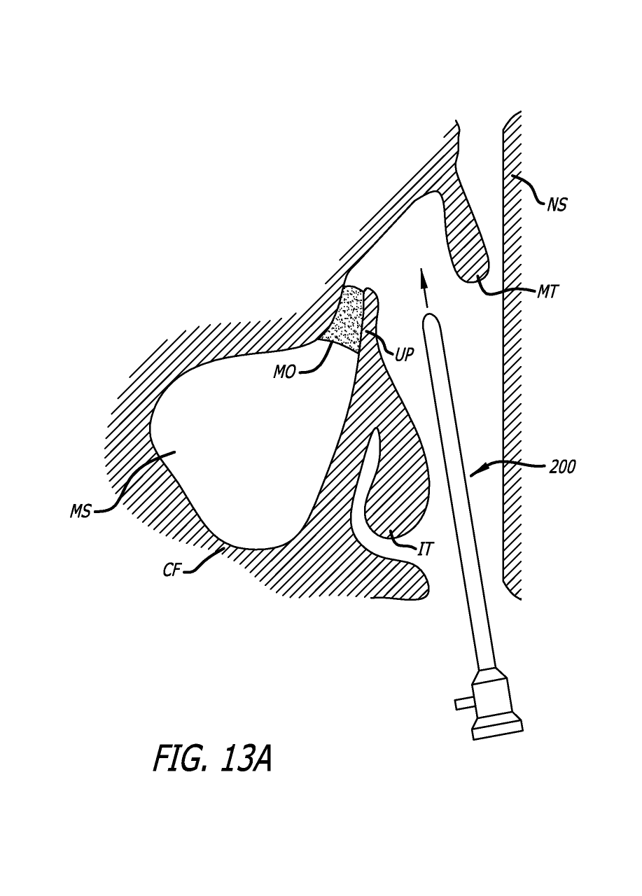

FIGS. 13A and 13B demonstrate, as an example, the use of a swing prism endoscope 200 in a transnasal (through the nostril) procedure. As shown in FIG. 13A, endoscope 200 may be used during insertion to look straight ahead, as ENT physicians are accustomed to doing in their current procedures with conventional rigid endoscopes. As in FIG. 13B, however, once endoscope 200 is sufficiently advanced, the viewing angle may be changed to look retrograde down the infundibulum into the maxillary sinus ostium MO. With the viewing angle so adjusted, endoscope 200 may be used to observe placement of a guide catheter 202, passage of a guidewire 206 out of guide catheter 202, passage of a balloon catheter 204 out of guide catheter 202 over guidewire 206, and expansion of balloon catheter 204 to dilate the ostium MO. In the past, endoscopes that could view at an angle could not also view straight ahead. This made their use very challenging and inconvenient for ENT physicians in particular. As described in U.S. Provisional Patent Application Ser. No. 61/084,949, however, swing prism endoscopes for viewing both straight ahead and at various angles have been invented, and these may be used in transnasal approaches, canine fossa access approaches or any other suitable use in the nasal and paranasal anatomy.

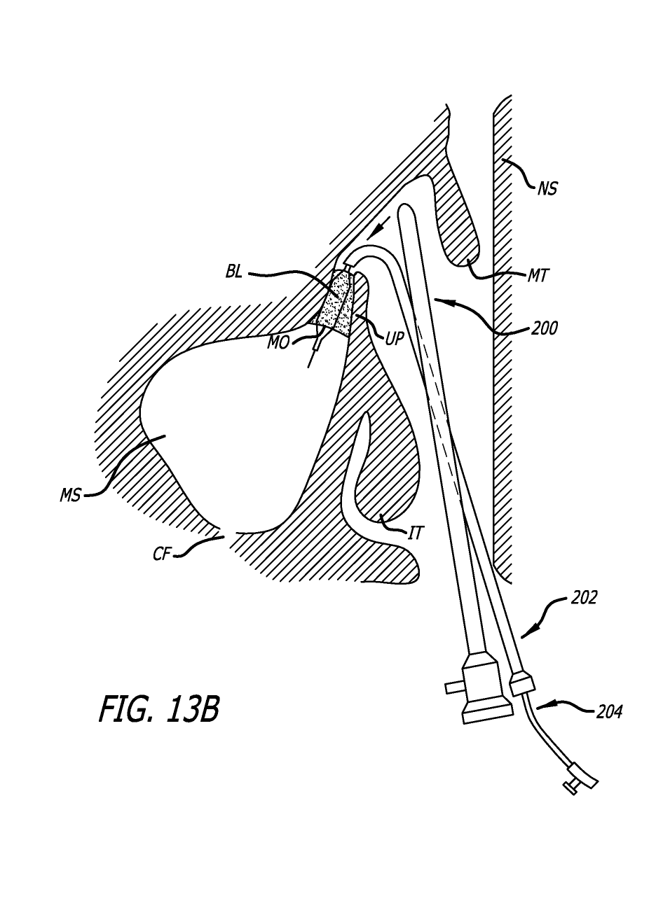

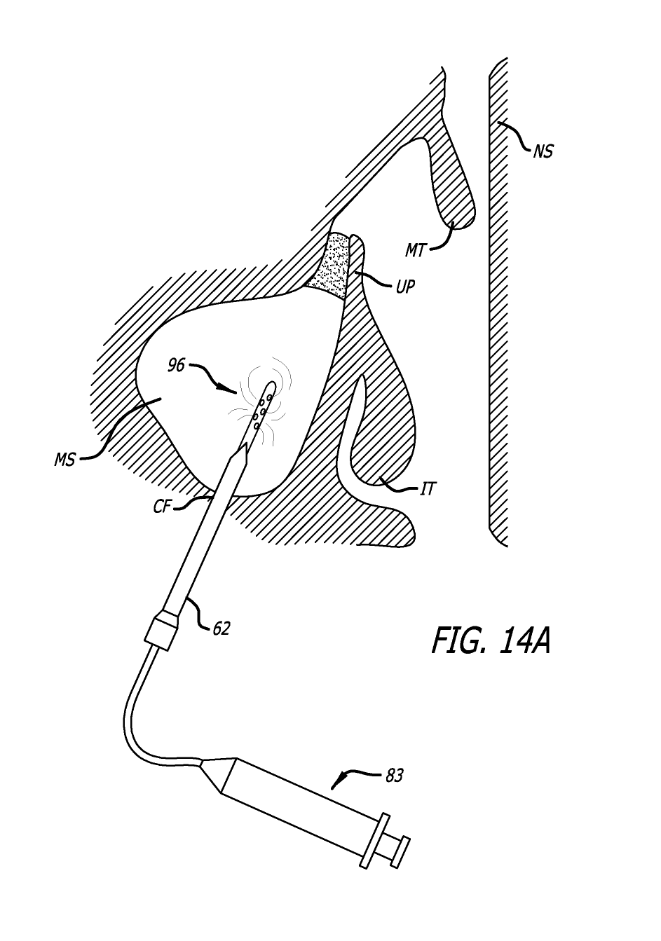

With reference now to FIG. 14A, another procedure that may be performed in a maxillary sinus MS through an opening created in a canine fossa CF is an irrigation (or “lavage”) procedure, using an irrigation catheter 82. In one embodiment, irrigation catheter 82 may comprise the Reliva Vortex™ Sinus Irrigation System (Acclarent, Inc., Menlo Park, Calif.). This sinus irrigation system and other embodiments are described in greater detail in U.S. patent Ser. No. 12/011,100, entitled Methods, Devices and Systems for Treatment and/or Diagnosis of Disorders of the Ear, Nose and Throat, and filed Jan. 24, 2008, the full disclosure of which is hereby incorporated by reference. In one embodiment, irrigation catheter 82 may include multiple irrigation ports near it tip in a pattern such that fluid ejected from catheter 82 creates a vortex configuration. Irrigation fluid may be provided, for example, via a syringe 83. The ejected fluid often helps to move collected mucus from the sinus and otherwise clean out the sinus. In various cases, mucus and irrigation fluid may drain out of the maxillary sinus ostium, the canine fossa puncture site, or both. In an alternative embodiment, a combined irrigation/suction device may be used. In another alternative embodiment, a suction device alone may be used to remove mucus and/or other fluids from the sinus.

In yet another alternative embodiment, and referring now to FIG. 14B, an access cannula 230 (or access sheath 232 or other canine fossa access devices in alternative embodiments) may include one or more built-in lumens for suction and/or irrigation. Thus, in this embodiment, suction and/or irrigation may be performed directly through cannula 230 without the need for a separate irrigation and/or suction catheter. Instead, an irrigation device 234 and a suction device 236 are directly attached to cannula 230, and fluid is pushed through and drawn back through irrigation and suction lumens, respectively. Of course, in alternative embodiments, cannula 230 or sheath 232 may have only irrigation lumen(s) or only suction lumen(s).

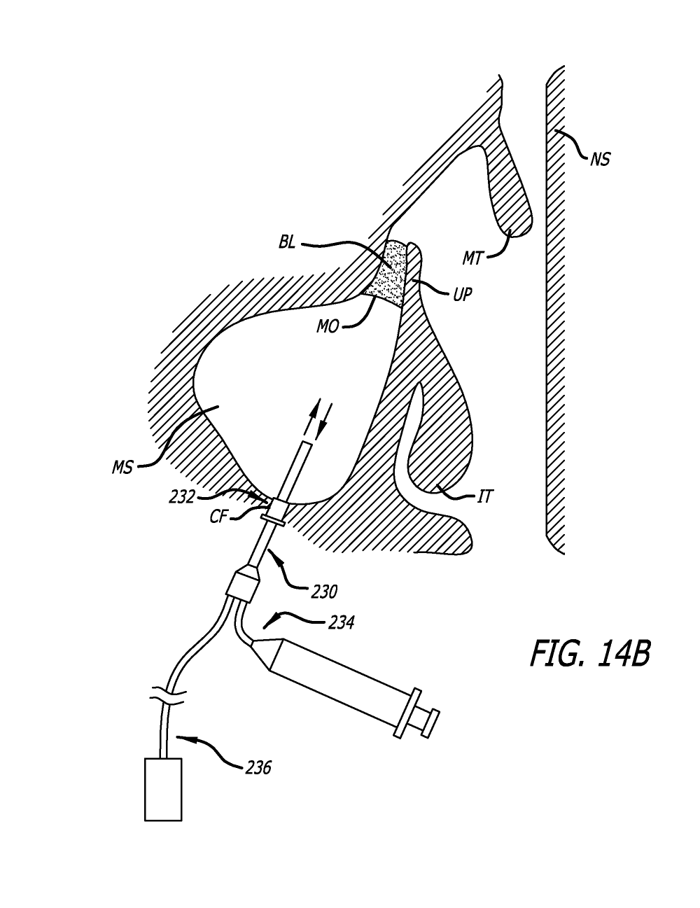

Referring now to FIG. 15, another example of a procedure that may be performed in a maxillary sinus MS via a canine fossa access approach, either by itself or in combination with a balloon dilation or other procedure, is tissue removal. In one embodiment, for example, a biopsy-like tissue removal device 180 may be advanced into the maxillary sinus MS through a cannula 62 or other access device positioned across the canine fossa CF to remove polyp tissue 182. Any suitable tissue removal device may be used, such as graspers, cutters, backbiters, scissors, radiofrequency ablation devices or the like. Optionally, multiple devices may be used. As shown, a distal portion of the tissue removal device 180 may include articulating opposing graspers or cutters, manipulated by a handle 181 or trigger mechanism to cut, grab and/or tear tissue. One type of tissue which may be removed is polyps, which may sometimes cause or exacerbate sinusitis.

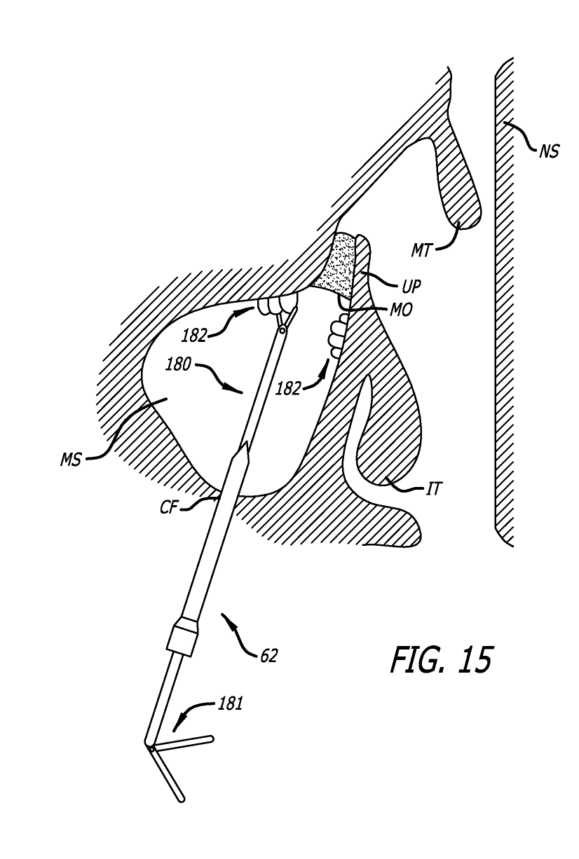

Turning now to FIG. 16, in another embodiment, a drug eluting sinus spacer device 192 may be delivered to a maxillary sinus through a canine fossa cannula 62 or other canine fossa access device. Drug eluting sinus spacers 192 and their use in paranasal sinuses are described in detail in U.S. Pat. Nos. 7,361,168, 7,410,480 and 7,419,497, as well as U.S. patent application Ser. No. 12/100,361, entitled Ethmoidotomy System and Implantable Spacer Having Therapeutic Substance Delivery Capability for Treatment of Paranasal Sinusitus, and filed Apr. 9, 2008, the full disclosures of which are hereby incorporated by reference. Generally, in some embodiments, sinus spacer 192 includes an expandable, drug eluting reservoir 194 with multiple micro-holes 195 for releasing drug, one or more retention wings 196 or other protrusions for helping spacer stay retained within a sinus after delivery, and a delivery catheter 191 for delivering spacer 192 to a sinus. Once delivered, a drug or combination of drugs, such as but not limited to steroidal anti-inflammatories, non-steroidal anti-inflammatories, antibiotics and/or the like may be delivered to reservoir 194 via catheter 191 and a syringe 193. Catheter 191 may then be detached from spacer 192 at a detachment point 198, and spacer 192 may be left in the sinus for any suitable length of time, such as but not limited to from 1 day to 180 days. During that time, drug slowly elutes from reservoir 194 through micro-holes 195. Spacer 192 may be removed by inserting a grasping device into the sinus and pulling it out of either the canine fossa hole (if it still exists) or the maxillary ostium. Wings 196 are configured to collapse when spacer 192 is pulled out of a sinus. One or more spacers 192 may be placed in any sinus or combination of sinuses, and any sinus may be accessed through a canine fossa CF approach by advancing a delivery catheter through the maxillary sinus ostium MO and directing toward another sinus. Again, for further detail regarding sinus spacers, reference may be made to the above-referenced patents and patent application.

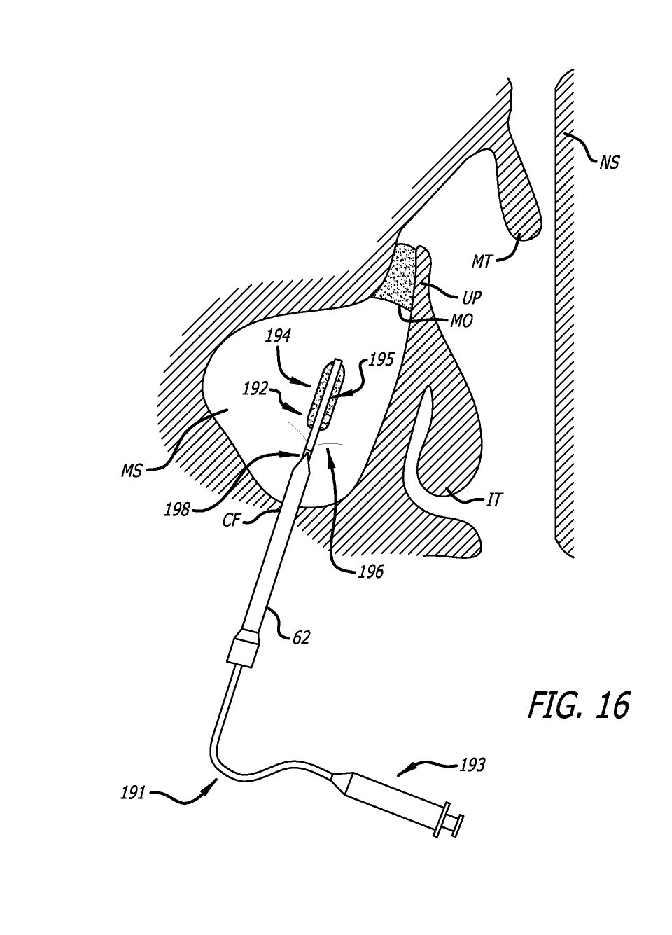

Referring to FIG. 17, another example of a procedure that may be performed across the canine fossa into the maxillary sinus is to fill the sinus with fluid via a needle/syringe 120 and acquire radiographic images of the sinus. For example, such images may include ultrasound, CT or MRI images. The presence of fluid may help enhance such imaging techniques. The same or a different needle/syringe 120 may also or alternatively be used to take a sample of fluid from a maxillary sinus for diagnostic testing.

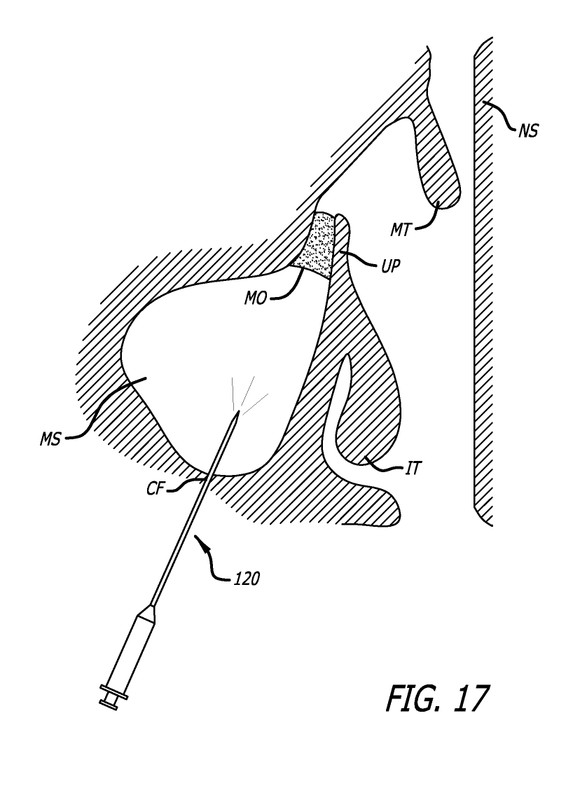

Referring now to FIG. 18, in another maxillary sinus procedure through a canine fossa access sheath 104, a balloon catheter 240 with a large, space-filling balloon 244 and an inflation device 242 may be used to drive mucus and/or other fluid out of the sinus via the maxillary ostium. In this procedure, balloon 244 is inflated sufficiently to consume a majority of the space within the sinus, thus driving out mucus and/or other fluids.

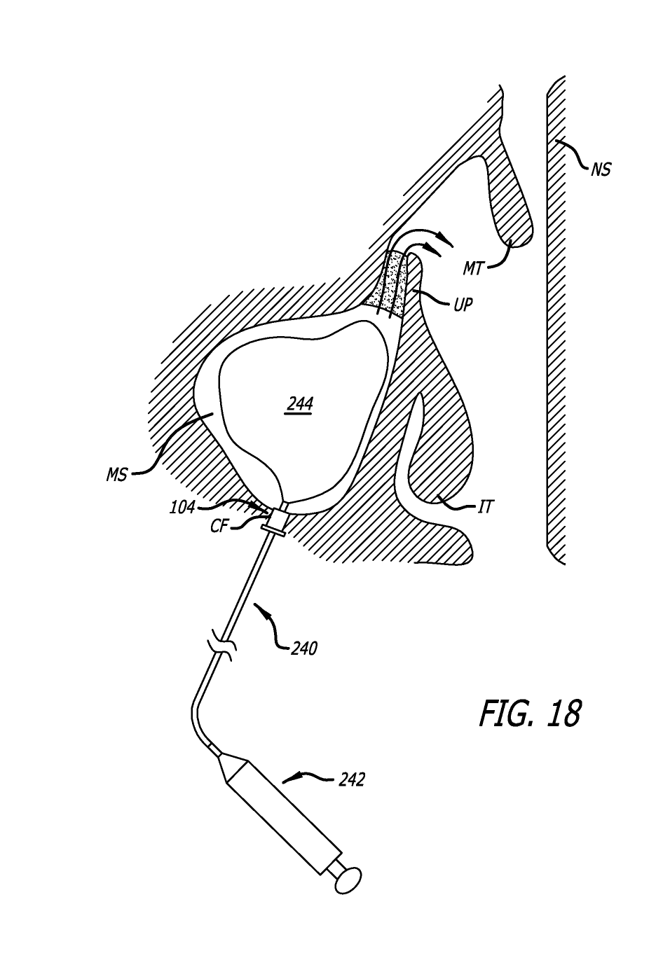

With reference now to FIGS. 19A and 19B, in one procedure balloon catheter 254 may be positioned in a maxillary sinus ostium by advancing it over or pulling it behind a guidewire 252 passed through the canine fossa and through the ostium. In one embodiment, guidewire 252 may be advanced through a guidewire lumen of a canine fossa access cannula 250 (or access sheath, cannula, needle, etc. in other embodiments) and thus through the maxillary sinus ostium MO. Balloon catheter 254 may be passed into the nostril and nasal cavity of the patient on the same side of the head. In some embodiments, as in FIG. 19A, a guide catheter 256 may be used to guide balloon catheter 254. Alternatively, in some embodiments it may be possible to forego guide catheter 256, as depicted in FIG. 19B. Balloon catheter 254 may then be coupled with guidewire 252. In one embodiment, as shown, catheter 254 may have a guidewire lumen and may be advanced over guidewire 252. In an alternative embodiment, guidewire 252 and/or catheter 254 may include attachment mechanism for attaching to one another, such as a small loop, lasso, hook or the like on the end of guidewire 252 and a respective attachment member on catheter 254. In such an embodiment, guidwire 252 may attach to catheter 254 and may be pulled proximally (or “retrograde”) to pull balloon catheter 254 into position in the maxillary sinus ostium. Once in place, catheter may be used to dilate the ostium.

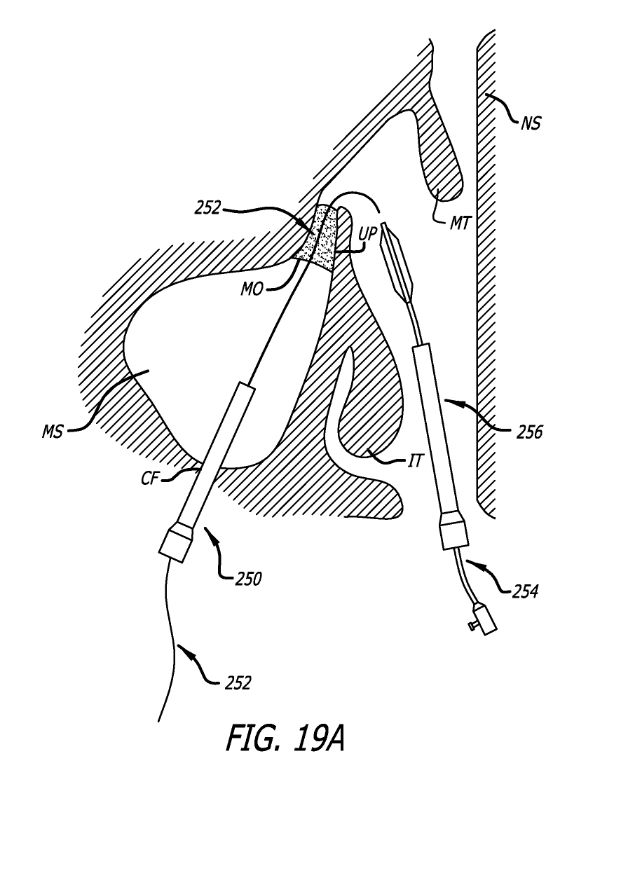

Referring now to FIGS. 20A and 20B, after one or more procedures have been performed on a maxillary sinus (and optionally one or more other sinuses) via a canine fossa puncture access approach, in one embodiment a mucosal tissue dressing 260 may be placed over the puncture site 262 to reduce bleeding, facilitate healing, reduce post-procedure pain, prevent fluid from passing into or out of the maxillary sinus, prevent infection of the sinus and/or the like. Tissue dressing 260 may be any suitable dressing, such as a one-layer or multi-layer dressing. In one embodiment, as shown in FIG. 20B, dressing 260 may include an adhesive layer 264 designed to adhere to mucosal tissue and lie directly over puncture site 262 and a barrier layer 266 immediately adjacent adhesive layer 264 for facing out toward the oral cavity. In some embodiment, either or both layers 264, 266 may contain one or more drugs, such as but not limited to an analgesic, an anti-inflammatory and/or an antibiotic. Various mucosal tissue dressings and methods for their use are described in greater detail in U.S. Provisional Patent Application Ser. No. 61/052,413, entitled Mucosal Tissue Dressing and Method of Use, and filed May 12, 2008, the full disclosure of which is hereby incorporated by reference.

As mentioned previously, most if not all of the procedures described above, as well as the post-procedure application of tissue dressing 260 may be applied not only to a canine fossa access approach to a maxillary sinus, but also to other approaches to the maxillary sinus and/or other approaches to other paranasal sinuses. For example, also described above was the trephine (or “mini-trephination”) approach to the frontal sinuses. Additional manmade puncture access techniques to the maxillary sinuses are described further below, and any of the procedures may be applied to those approaches. Furthermore, multiple sinuses may be accessed from one access site, such as when canine access puncture is used to access first a maxillary sinus and subsequently an ethmoid, frontal and/or sphenoid sinus. Therefore, although the foregoing description and accompanying drawings have focused on a canine fossa access approach and treatment methods for use in the maxillary sinuses, these are but examples of ways the procedures may be performed, and most if not all the embodiments may be alternatively or additionally applied to other paranasal sinuses.

Alternative Access Approaches to a Maxillary Paranasal Sinus

The assignee of the present invention has previously described techniques, devices and systems for accessing the paranasal sinuses (including the maxillary sinuses) transnasally (i.e., through the nostril directly to the sinus ostium). For example, reference may be made to U.S. patent application Ser. Nos. 10/829,917, 10/944,270, 11/037,548,11/150,847 and 11/193,020, the full disclosures of which are hereby incorporated by reference. Methods and devices for accessing the maxillary sinuses through a canine fossa piercing approach are described above. It may also be possible and perhaps advantageous in some cases to access a maxillary sinus through an alternative to the transnasal and canine fossa approaches. The following describes some examples of such alternative approaches.

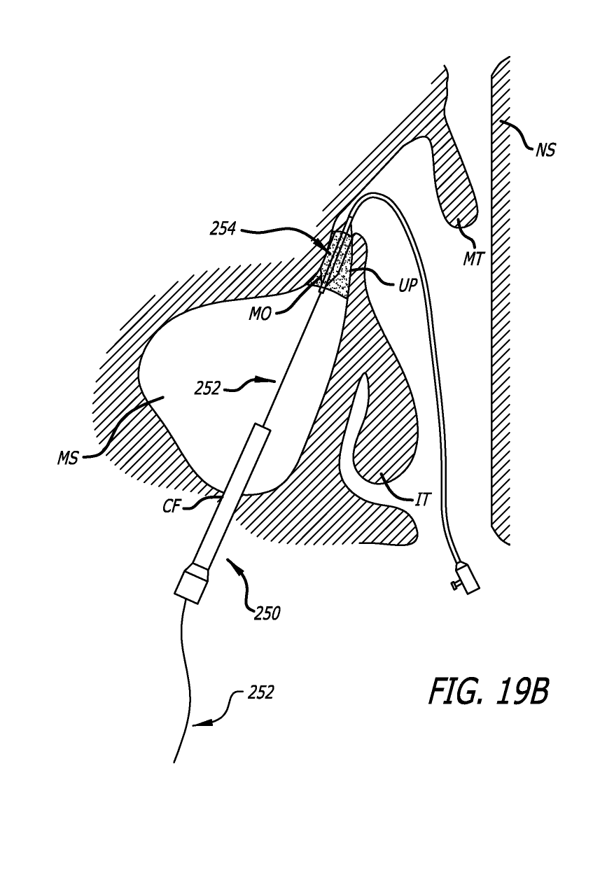

Referring now to FIG. 21, in one embodiment, an access cannula 400 or other access device may be used to access a maxillary sinus MS by advancement through a nostril and then through an opening in the inferior meatus IM of the nasal cavity (the region under/inferior to the inferior turbinate IT). (Neither access cannula 400 nor many of the devices in the above-described and following drawings is drawn to scale.) In some embodiments, this access route may be anterior to the lacrimal duct (not shown). In various embodiments, any of the access devices described above or in the patent applications incorporated by reference may be used.

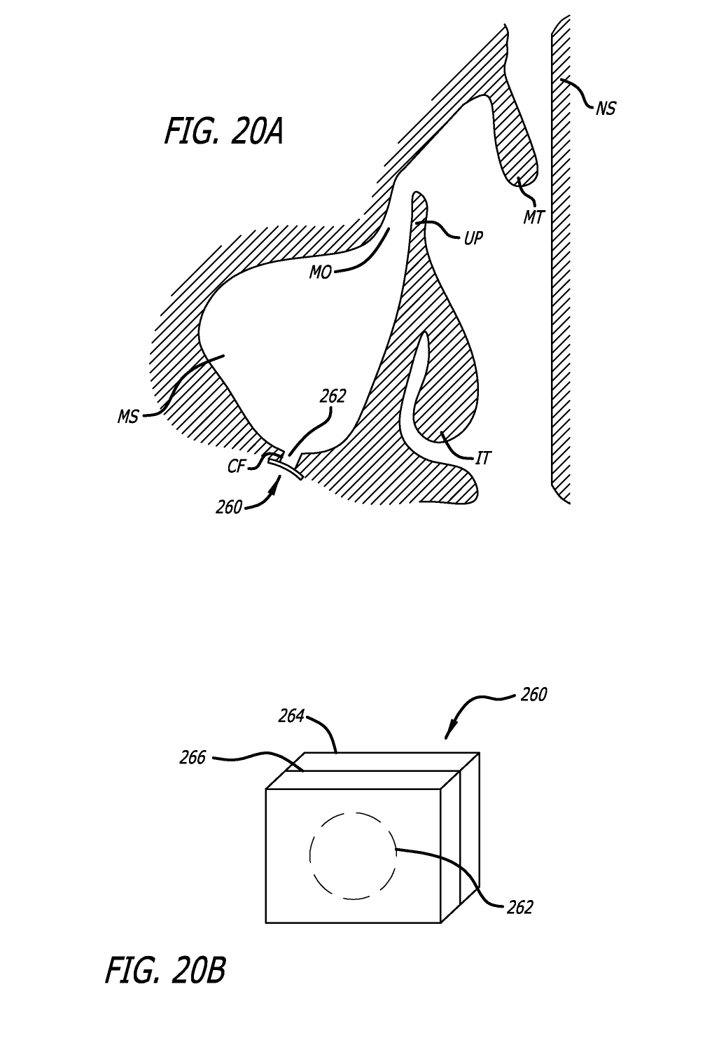

With reference now to FIGS. 22A and 22B, in an alternative embodiment, an access cannula 402 or other access device may be used to access a maxillary sinus (not visible in these figures) by advancing into a nostril and piercing an opening through the uncinate process UP into the sinus. The uncinate process UP is removed in FIG. 22A to show only the connection of the uncinate process UP to the lateral nasal wall. Piercing directly through the uncinate process UP makes it unnecessary to navigate a curved guide around the uncinate process to access the maxillary sinus ostium MO.

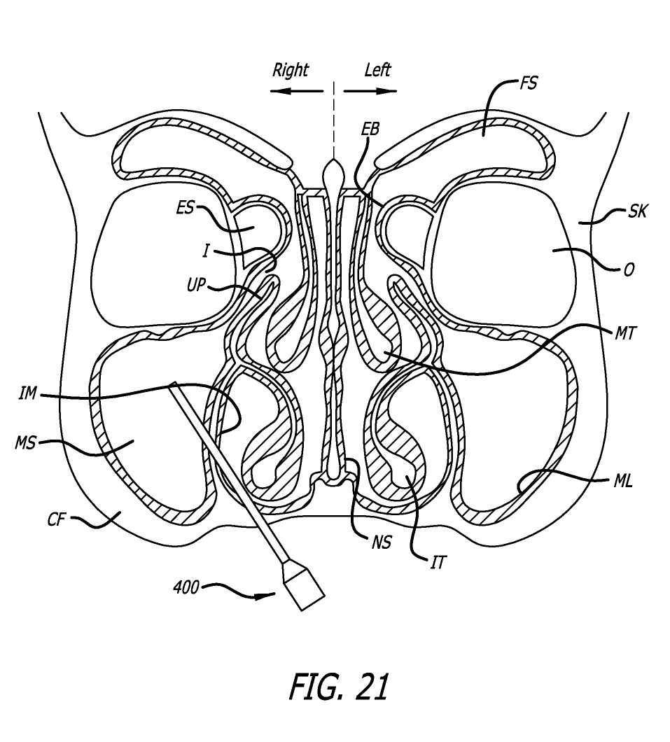

FIG. 23 demonstrates another method for accessing a maxillary sinus through a physician created opening 412 in an uncinate process UP. In this embodiment, an endoscope 410 is advanced through manmade opening 412, a guide 414 is advanced into the nostril and nasal cavity, and a guidewire 416 is advanced out of the guide into the maxillary sinus MS through the maxillary ostium. The endoscope is advantageous for the physician positioned in this manner because it facilitates a safer and more accurate operating procedure, because the devices moving through and action on the ostium can be moved from the side and out in front of them. It also is better for the patient, because a fluoroscopy system does not need to be used so there is no radiation exposure. This embodiment illustrates that for any given procedure, access to a paranasal sinus may be achieved by a combination of access routes with a combination of instruments. In this case, access is gained both transnasally through the ostium and transnasally through an opening in the uncinate process. In other embodiments, the endoscope can be inserted through a physician created opening in the canine fossa, palate, etc. as previously discussed. In various embodiments, any of the access routes described in this application and any of the access and/or procedural devices may be used in any combination.

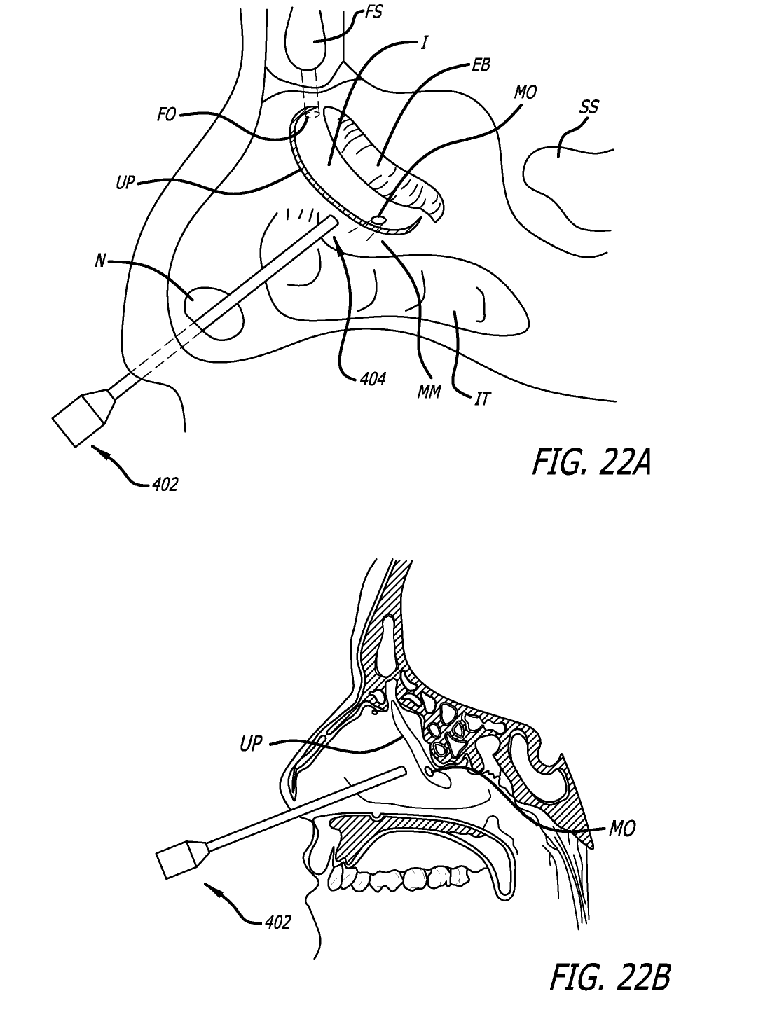

Referring to FIG. 24, another route for accessing a maxillary sinus MS is with an access device 420 advanced through the mouth and through an opening in the palate P. Again, any device may be placed using this access route, and in some cases a combination access procedure may be used, such as in combination with a device placed transnasally.

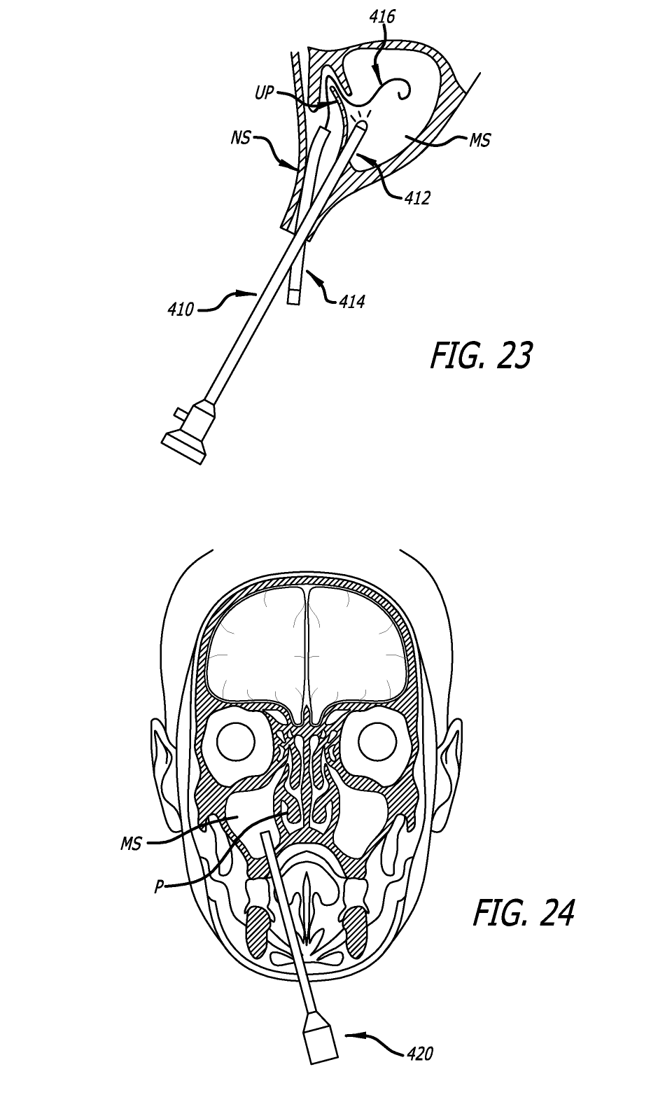

FIG. 25 illustrates another access device 430, placed through the nostril and extending through an opening in the posterior fonatanelle PF. This access route may utilize a curved access device 430.

As previously mentioned, any of the foregoing access devices and methods described in this application may be used in combination or in the alternative in various embodiments.

Alternative Balloon Designs

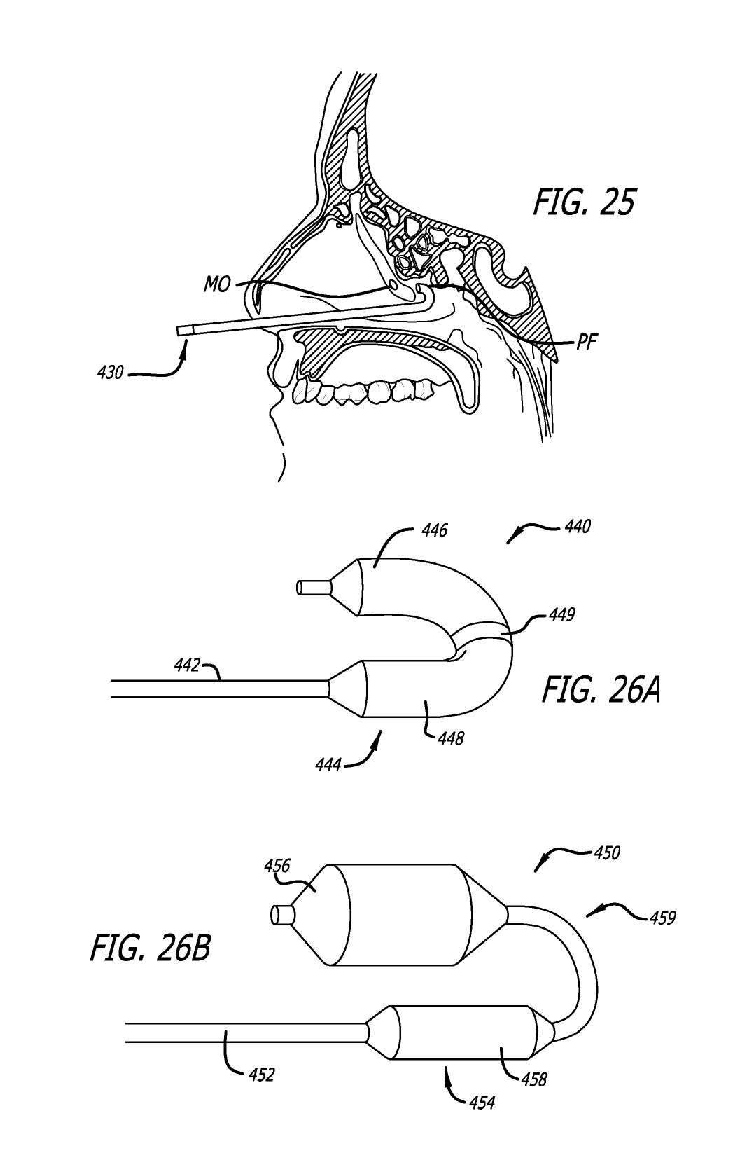

With reference now to FIGS. 26A and 26B, alternative balloon shapes for balloon catheters may sometimes be desirable in dilating paranasal sinus ostia using a transnasal access approach or any of the access approaches described above. The balloon shapes illustrated in FIGS. 26A and 26B may be particularly advantageous for dilating maxillary sinus ostia using a transnasal access approach.

FIG. 26A shows a distal portion of a balloon catheter 440 having a shaft 442 and a balloon 444. Balloon 444 includes a distal maxillary ostium region 446, a proximal middle meatus region 448, and a curve 449 between the two. This shape of balloon 444 allows curve 449 to wrap over/around an uncinate process, with maxillary region 446 residing in the maxillary sinus ostium and middle meatus region 448 contacting the middle meatus. The advantages of such a shape may include that the uncinate process is protected from damage during a procedure, both the ostium and the metal region are dilated during the procedure, and the shape may help anchor balloon 444 to prevent “watermelon seeding” of the balloon out of the ostium during balloon dilation. (Watermelon seeding refers to the phenomenon of a balloon slipping out of an ostium during inflation, typically due to the balloon being asymmetrically positioned relative to the ostium before inflation.)

FIG. 26B shows an alternative embodiment of a distal portion of a balloon catheter 450, again including a catheter shaft 452 and a balloon 454. In this embodiment, balloon 454 includes different diameters in its meatus region 458, maxillary region 456 and curved portion 459. In some embodiments, maxillary region 456 may be more compliant than meatus region 458. Maxillary region 456 may also have a larger diameter than meatus region 458. Balloon 454 in this configuration confers similar advantages to balloon 444 in FIG. 26A.

The various embodiments described above are provided by way of illustration only and should not be construed to limit the scope of the invention as it is set forth in the following claims. Various modifications and changes may be made to the disclosed embodiments without departing from the spirit and scope of the invention. For example, in various alternative embodiments, method steps described above may be interchanged, added or deleted. Devices used in one method may be used in an alternative method, and devices described in terms of one system may be combined with different devices in an alternative system. Therefore, the foregoing description should not be interpreted to limit the scope of the invention described in the claims.

What is claimed:

1. A method for dilating a maxillary paranasal sinus ostium in a patient, the method comprising:
forming an opening into a maxillary sinus through a canine fossa of a patient with a tissue-piercing, dual-lumen cannula;
advancing a balloon catheter out of a distal opening in a first lumen of the cannula;
viewing the balloon catheter using an endoscope disposed in a second lumen of the cannula;
expanding a balloon of the balloon catheter within an ostium of the maxillary sinus to dilate the ostium, causing breakage or rearrangement of bone underlying mucosa of the ostium, while viewing the expansion using the endoscope;
deflating the balloon;
advancing the balloon through the maxillary sinus ostium; and
expanding the balloon in another area inside the patient's head to dilate that area.

2. The method of claim 1, wherein the deflated balloon is advanced into an osteomeatal complex of the patient and the osteomeatal complex is dilated.

3. The method of claim 1, wherein the deflated balloon is advanced into a frontal sinus outflow tract or a frontal sinus ostium of the patient and the frontal sinus outflow tract or frontal sinus ostium is dilated.

4. The method of claim 1, wherein forming the opening comprises:
piercing through the canine fossa with a sharp-tipped piercing device disposed within one of lumens of the cannula; and
removing or retracting the piercing device through the cannula.

5. The method of claim 1, further comprising, before advancing the balloon catheter, advancing a guidewire into the maxillary sinus, wherein the balloon catheter is advanced over the guidewire.

6. The method of claim 5, wherein the guidewire comprises a light emitting guidewire, the method further comprising:
emitting light from the light emitting guidewire into the maxillary sinus; and
viewing the emitting light from outside the patient, through the patient's skin, to confirm positioning of the guidewire in the maxillary sinus.

7. The method of claim 1, further comprising advancing a distal end of the endoscope out of a distal opening in the second lumen.

8. The method of claim 7, further comprising coupling a proximal end of the endoscope with the patient or an object outside the patient to secure the endoscope during the procedure.

9. The method of claim 1, further comprising:
removing the balloon catheter from the maxillary sinus through the cannula;
advancing an irrigation catheter through the first lumen into the maxillary sinus; and
irrigating the maxillary sinus using the irrigation catheter.

10. The method of claim 1, further comprising:
removing the balloon catheter from the maxillary sinus through the cannula;
advancing a tissue removal device through the first lumen into the maxillary sinus; and
removing tissue from the maxillary sinus using the tissue removal device.

11. A method for treating a paranasal sinus in a patient, the method comprising:
forming an opening through a canine fossa into a maxillary paranasal sinus; and
performing at least one procedure other than a balloon dilation procedure within the maxillary sinus or within another paranasal sinus of the patient's head, using a device advanced through the opening in the canine fossa.

12. The method of claim 11, further comprising advancing a guide through the opening in the canine fossa, wherein the device is advanced into the maxillary or other sinus over or through the guide.

13. The method of claim 11, wherein performing the procedure comprises:
injecting fluid through the canine fossa into the maxillary sinus; and
visualizing the maxillary sinus using an imaging method selected from the group consisting of ultrasound, computed tomography and magnetic resonance imaging.

14. The method of claim 11, wherein performing the procedure comprises irrigating the maxillary sinus using a sinus irrigation catheter advanced through the canine fossa opening.

15. The method of claim 11, wherein performing the procedure comprises suctioning substance from the maxillary sinus using a suction catheter advanced through the canine fossa opening.

16. The method of claim 11, wherein performing the procedure comprises inflating a large balloon within the maxillary sinus to push mucus out of the sinus.

17. The method of claim 11, wherein performing the procedure comprises removing polyp tissue from inside the maxillary sinus using a tissue removal device advanced through the canine fossa opening.

18. The method of claim 11, wherein performing the procedure comprises:
advancing a drug eluting sinus spacer through the canine fossa opening and into the maxillary sinus, a frontal sinus, a frontal sinus outflow tract, an ethmoid sinus or a sphenoid sinus; and
leaving the sinus spacer in place for a period of between 1 and 180 days.

19. The method of claim 11, further comprising placing a one-way valve in the canine fossa opening, wherein the procedure is performed through the valve.

20. The method of claim 11, further comprising:
inserting a dilator device into the opening in the canine fossa; and
using the dilator to dilate or expand the opening.

21. The method of claim 11, further comprising using a nerve detection device to detect at least one nerve adjacent the canine fossa prior to forming the canine fossa opening.

22. The method of claim 11, further comprising:
advancing an endoscope into the maxillary sinus through the canine fossa opening; and
visualizing at least one of the device or an inner wall of the maxillary sinus using the endoscope.

23. The method of claim 23, further comprising changing a viewing angle of the endoscope by adjusting a swing prism in the endoscope.

24. The method of claim 11, further comprising performing a balloon dilation procedure, comprising:
inserting a balloon catheter through the canine fossa opening into the maxillary sinus;
advancing the balloon catheter to position a balloon of the catheter in a maxillary sinus ostium; and
expanding the balloon to dilate the maxillary sinus ostium, causing breakage or rearrangement of bone underlying mucosa of the ostium.

25. The method of claim 24, further comprising:
deflating the balloon;
advancing the balloon catheter through the maxillary sinus ostium and into a frontal sinus outflow tract or frontal sinus ostium;
expanding the balloon to dilate the frontal sinus outflow tract or frontal sinus ostium, causing breakage or rearrangement of bone underlying mucosa of the ostium;
deflating the balloon; and
removing the balloon catheter through the canine fossa opening.

26. The method of claim 24, further comprising, before advancing the balloon catheter, advancing a guide into the maxillary sinus through the canine fossa opening, wherein the balloon catheter is advanced over or through the guide.

27. The method of claim 26, wherein the guide comprises an illuminating guidewire, the method further comprising:
emitting light from the guidewire at or near its distal end; and
viewing the emitted light from outside the patient to confirm positioning of the guidewire distal end in the maxillary sinus.

28. The method of claim 24, wherein the guide comprises a cannula, and wherein the catheter is advanced through a lumen of the cannula.

29. The method of claim 28, wherein forming the opening through the canine fossa comprises piercing the canine fossa with a piercing member coupled with the cannula.

30. A method for treating a maxillary paranasal sinus in a patient, the method comprising:
forming an opening through a canine fossa into a maxillary paranasal sinus;
advancing a distal end of a guidewire through the opening and through a maxillary sinus ostium, into a nostril of the patient's head;
advancing a balloon catheter into the nostril;
coupling the balloon catheter with the distal end of the guidewire;
advancing the balloon catheter into the maxillary sinus ostium using the guidewire; and
expanding the balloon to dilate the maxillary sinus ostium.

31. The method of claim 30, wherein coupling the balloon catheter with the distal end of the guidewire comprises attaching a first attachment member disposed on the catheter with a second attachment member disposed on the guidewire, and wherein the catheter is advanced into the ostium by pulling the guidewire back through the ostium.

32. The method of claim 30, wherein coupling the balloon catheter with the distal end of the guidewire comprises advancing the catheter over the guidewire.

33. The method of claim 30, further comprising viewing the balloon catheter in the nostril using an endoscope positioned in the nostril.

34. The method of claim 30, further comprising viewing a distal end of the balloon catheter in the maxillary sinus ostium using an endoscope positioned in the maxillary sinus via the canine fossa opening.

35. A method for dilating a maxillary paranasal sinus ostium, the method comprising:
forming an opening through a maxillary sinus wall into a maxillary sinus in an area not at the canine fossa;
advancing a flexible balloon catheter through the opening to position a balloon of the catheter in the maxillary sinus ostium; and
expanding the balloon to dilate the maxillary sinus ostium, causing breakage or rearrangement of bone underlying mucosa of the ostium.

36. The method of claim 35, wherein the area where the opening is formed is selected from the group consisting of an inferior meatus, a palate, an uncinate process and a posterior fontanelle.

37. The method of claim 36, wherein the opening is formed anterior to a lacrimal duct in the nasal cavity.

38. The method of claim 35, further comprising, before advancing the catheter, advancing a guide device into the nasal cavity to a position in or near the opening, wherein the catheter is advanced over or through the guide device.

39. The method of claim 35, further comprising viewing at least part of the method using an endoscope advanced through a nostril of the patient.

40. A system for treating a maxillary paranasal sinus, the system comprising:
a tissue-piercing cannula for forming an opening through a canine fossa into a maxillary paranasal sinus, wherein the cannula includes at least two lumens for guiding at least two devices into the maxillary sinus;
a balloon catheter configured to be inserted through a first lumen of the cannula; and
an endoscope configured to be inserted through a second lumen of the cannula.

41. The system of claim 40, wherein the cannula comprises a sharp tip, the system further comprising at least one lumen filling member disposed within the lumens during advancement of the cannula through the canine fossa.

42. The system of claim 40, wherein the cannula comprises a blunt tip, the system further comprising at least one sharp-tipped device removably disposed within at least one of the lumens for forming the opening through the canine fossa.

43. The system of claim 40, further comprising at least one additional device insertable through at least one of the lumens of the cannula after removal of the balloon catheter or the endoscope, the additional device selected from the group consisting of a tissue removal tool, a suction catheter, an irrigation catheter, a combined suction/irrigation catheter, an illuminating guidewire, a non-illuminating guidewire and a drug eluting sinus spacer.

44. The system of claim 40, wherein the cannula has an angled portion configured to direct the balloon catheter toward an ostium of the maxillary paranasal sinus.

45. The system of claim 40, wherein the balloon catheter has a length sufficient to extend from outside the patient, through a maxillary sinus ostium, and to a frontal sinus outflow tract or frontal sinus ostium.

46. The system of claim 40, further including at least one mucosal tissue dressing for covering the opening formed through the canine fossa after the cannula is removed.

47. A system for treating a maxillary paranasal sinus, the system comprising:
a piercing device for piercing an opening through a canine fossa into a maxillary paranasal sinus;
a one-way valve configured for placement in the opening; and
a balloon catheter configured to be inserted through the one-way valve.

48. The system of claim 47, further comprising at least one additional device insertable through the one-way valve, the additional device selected from the group consisting of an endoscope, a tissue removal tool, a suction catheter, an irrigation catheter, a combined suction/irrigation catheter, an illuminating guidewire, a non-illuminating guidewire and a drug-eluting sinus spacer.
